(19) United States
(12) Patent Application Publication
(10) Pub. No.: US 2006/0003927 A1
(43) Pub. Date: Jan. 5, 2006

(54) Modulation of immune function

(76) Inventors: Brian Robert Champion, Cambridge (GB); Lesley Lynn Young, Cambridge (GB)

Correspondence Address: FROMMER LAWRENCE & HAUG, 745 FIFTH AVENUE- 10TH FL., NEW YORK, NY, 10151, US

(21) Appl. No.: 11/103,077

(22) Filed: Apr. 11, 2005

Related U.S. Application Data
(63) Continuation-in-part of application No. PCT/GB03/04402, filed on Oct. 9, 2003.

(30) Foreign Application Priority Data
Oct. 9, 2002 (GB) 0223405.2
Oct. 9, 2002 (GB) 0223409.4
Oct. 19, 2002 (GB) 0224353.3

(51) Int. Cl. A61K 38/13; A61K 31/573; A61K 31/4745

(52) U.S. Cl. 514/11; 514/171; 514/291

(57) ABSTRACT

A method for modulating the immune system in a mammal is described comprising simultaneously, contemporaneously, separately or sequentially administering: i) an effective amount of an agonist of Notch signalling; and ii) an effective amount of an immunosuppressive agent.

### CROSS REFERENCE TO RELATED APPLICATIONS

This application is a continuation-in-part of International Application No. PCT/GB2003/004402, filed on Oct. 9, 2003, published as WO 2004/032969 on Apr. 22, 2004, and claiming priority to GB Application Serial Nos. 0223405.2 and 0223409.4, both filed Oct. 9, 2002, and 0224353.3, filed Oct. 19, 2002.

Reference is made to U.S. application Ser. No. 09/310,685, filed May 4, 1999; Ser. No. 09/870,902, filed May 31, 2001; Ser. No. 10/013,310, filed Dec. 7, 2001; Ser. No. 10/147,354, filed May 16, 2002; Ser. No. 10/357,321, filed Feb. 3, 2002; Ser. No. 10/682,230, filed Oct. 9, 2003; Ser. No. 10/720,896, filed Nov. 24, 2003; Ser. Nos. 10/763,362, 10/764,415 and 10/765,727, all filed Jan. 23, 2004; Ser. No. 10/812,144, filed Mar. 29, 2004; Ser. Nos. 10/845,834 and 10/846,989, both filed May 14, 2004; Ser. No. 10/877,563, filed Jun. 25, 2004; Ser. No. 10/899,422, filed Jul. 26, 2004; Ser. No. 10/958,784, filed Oct. 5, 2004; Ser. No. 11/050,328, filed Feb. 3, 2005; Ser. No. 11/058,066, filed Feb. 14, 2005; Ser. No. 11/071,796, filed Mar. 3, 2005; and Ser. No. 11/078,735, filed Mar. 10, 2005.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention. Documents incorporated by reference into this text are not admitted to be prior art.

### FIELD OF THE INVENTION

The present invention relates to the modulation of immune function.

### BACKGROUND OF THE INVENTION

International Patent Publication No WO 98/20142 describes how manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases may be targeted.

It has also been shown that it is possible to generate a class of regulatory T cells which are able to transmit antigen-specific tolerance to other T cells, a process termed infectious tolerance (WO98/20142).

A description of the Notch signalling pathway and conditions affected by it may be found, for example, in our published PCT Applications as follows:
PCT/GB97/03058 (filed on 6 Nov. 1997 and published as WO 98/20142; claiming priority from GB 9623236.8 filed on 7 Nov. 1996, GB 9715674.9 filed on 24 Jul. 1997 and GB 9719350.2 filed on 11 Sep. 1997);
PCT/GB99/04233 (filed on 15 Dec. 1999 and published as WO 00/36089; claiming priority from GB 9827604.1 filed on 15 Dec. 1999);
PCT/GB00/04391 (filed on 17 Nov. 2000 and published as WO 0135990; claiming priority from GB 9927328.6 filed on 18 Nov. 1999);
PCT/GB01/03503 (filed on 3 Aug. 2001 and published as WO 02/12890; claiming priority from GB 0019242.7 filed on 4 Aug. 2000);
PCT/GB02/02438 (filed on 24 May 2002 and published as WO 02/096952; claiming priority from GB 0112818.0 filed on 25 May 2001);
PCT/GB02/03381 (filed on 25 Jul. 2002 and published as WO 03/012111; claiming priority from GB 0118155.1 filed on 25 Jul. 2001);
PCT/GB02/03397 (filed on 25 Jul. 2002 and published as WO 03/012441; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);
PCT/GB02/03426 (filed on 25 Jul. 2002 and published as WO 03/011317; claiming priority from GB0118153.6 filed on 25 Jul. 2001, GB0207930.9 filed on 5 Apr. 2002, GB 0212282.8 filed on 28 May 2002 and GB 0212283.6 filed on 28 May 2002);
PCT/GB02/04390 (filed on 27 Sep. 2002 and published as WO 03/029293; claiming priority from GB 0123379.0 filed on 28 Sep. 2001);
PCT/GB02/05137 (filed on 13 Nov. 2002 and published as WO 03/041735; claiming priority from GB 0127267.3 filed on 14 Nov. 2001, PCT/GB02/03426 filed on 25 July 2002, GB 0220849.4 filed on 7 Sep. 2002, GB 0220913.8 filed on 10 Sep. 2002 and PCT/GB02/004390 filed on 27 Sep. 2002);
PCT/GB02/05133 (filed on 13 Nov. 2002 and published as WO 03/042246; claiming priority from GB 0127271.5 filed on 14 Nov. 2001 and GB 0220913.8 filed on 10 Sep. 2002).

Each of PCT/GB97/03058 (WO 98/20142), PCT/GB99/04233 (WO 00/36089), PCT/GB00/04391 (WO 0135990), PCT/GB01/03503 (WO 02/12890), PCT/GB02/02438 (WO 02/096952), PCT/GB02/03381 (WO 03/012111), PCT/GB02/03397 (WO 03/012441), PCT/GB02/03426 (WO 03/011317), PCT/GB02/04390 (WO 03/029293), PCT/GB02/05137 (WO 03/041735) and PCT/GB02/05133 (WO 03/042246) is hereby incorporated herein by reference

Reference is made also to Hoyne G. F. et al (1999) Int Arch Allergy Immunol 118:122-124; Hoyne et al. (2000) Immunology 100:281-288; Hoyne G. F. et al (2000) Intl Immunol 12:177-185; Hoyne, G. et al. (2001) Immunological Reviews 182:215-227; each of which is also incorporated herein by reference.

The present invention seeks to provide further methods of modulating the immune system.

Immunophilin-binding immunosuppressive agents such as rapamycin, cyclosporin(e) A and FK506 are known to show immunosuppressive effects and are thus commonly used for the treatment of conditions such as graft rejection.

Immunologically active steroids are known to show immunosuppressive effects and are commonly used, for example, for the treatment of inflammatory disorders.

It has now been found that the immunosuppressive effects of such immunosuppressive agents do not adversely interfere with the immunomodulatory action of modulators of the Notch signalling pathway, and moreover that combinations of such agents may be used to provide an unexpectedly improved spectrum and/or selectivity of activity, especially with reference to cytokine profiles.

### SUMMARY OF THE INVENTION

According to a first aspect of the invention there is provided a product comprising a modulator of the Notch signalling pathway (also referred to herein as a “modulator of Notch signalling”) and an immunosuppressive agent as a combined preparation for simultaneous, contemporaneous, separate or sequential use for modulation of the immune system.

According to a further aspect of the invention there is provided a method of modulating the immune system in a mammal comprising simultaneously, contemporaneously, separately or sequentially administering to a mammal in need thereof an effective amount of a modulator of the Notch signalling pathway and an effective amount of an immunosuppressive agent.

According to a further aspect of the invention there is provided a combination of a modulator of the Notch signalling pathway and an immunosuppressive agent; for simultaneous, contemporaneous, separate or sequential use in modulating the immune system.

According to a further aspect of the invention there is provided a modulator of the Notch signalling pathway for use in modulating the immune system in simultaneous, contemporaneous, separate or sequential combination with an immunosuppressive agent.

According to a further aspect of the invention there is provided the use of a combination of a modulator of the Notch signalling pathway and an immunosuppressive agent; in the manufacture of a medicament for modulation of the immune system.

According to a further aspect of the invention there is provided the use of a modulator of the Notch signalling pathway in the manufacture of a medicament for modulation of the immune system in simultaneous, contemporaneous, separate or sequential combination with an immunosuppressive agent.

According to a further aspect of the invention there is provided a kit comprising a modulator of the Notch signalling pathway and an immunosuppressive agent.

According to a further aspect of the invention there is provided a method for modulation of the immune system comprising contacting a naive T cell with a stimulatory signal and an appropriate amount of a combination of a modulator of Notch signalling and an immunosuppressive agent, to promote differentiation to a regulatory T cell.

The methods, products and uses of the present invention provide enhanced biological or therapeutic effects. The term “enhanced biological or therapeutic effects” as used herein includes, for example, increased potency, increased efficacy, decreased side effects, improved activity spectrum, and the like.

In one preferred embodiment, the immunosuppressive agent is an immunophilin-binding immunosuppressive agent.

In an alternative preferred embodiment, the immunosuppressive agent is an immunologically active steroid.

In one embodiment the modulator of the Notch signalling pathway and the immunophilin-binding immunosuppressive agent or the immunologically active steroid are used in synergistic (suitably in superadditive) amounts. This has the particular advantage that lower amounts of active agents may be used if desired.

According to a further aspect of the invention there is provided a method for modulating the immune system, comprising the steps of administering (in any order) an effective amount of a modulator of Notch signalling in a first treatment procedure; and administering an effective amount of an immunosuppressant agent in a second treatment procedure.

According to a further aspect of the invention there is provided a method for modulating the immune system, comprising the steps of administering (in any order) a synergistically effective amount of a modulator of Notch signalling in a first treatment procedure; and administering a synergistically effective amount of an immunosuppressant agent in a second treatment procedure.

Suitably an immunophilin-binding immunosuppressive agent may act by binding to cyclophilin.

Alternatively an immunophilin-binding immunosuppressive agent may act by binding to macrophilin/FKBP.

Alternatively an immunophilin-binding immunosuppressive agent may act by binding to mTOR or RAPTOR.

Suitably an immunophilin-binding immunosuppressive agent comprises a rapamycin or a rapamycin derivative.

Alternatively or in addition an immunophilin-binding immunosuppressive agent comprises a cyclosporin or a cyclosporin derivative.

Alternatively or in addition an immunophilin-binding immunosuppressive agent comprises FK506 (tacrolimus) or an FK506 derivative.

Preferably the modulator of the Notch signalling pathway (“modulator of Notch signalling”) is an agent capable of activating Notch signalling. Preferably the agent is capable of activating Notch signalling in lymphocytes, preferably in T-cells.

Preferably the modulator of the Notch signalling pathway is an agent capable of activating a Notch receptor, such as a Notch1, Notch2, Notch3 or Notch4 receptor. Suitably, for example, the modulator may be a Notch ligand or a biologically active fragment or derivative of a Notch ligand, or a peptidomimetic of such a Notch ligand. Preferably the agent is capable of activating Notch receptors in lymphocytes such as T-cells, preferably in mammals, preferably in humans.

Suitably the modulator of the Notch signalling pathway may comprise or code for a fusion protein. For example, the modulator may comprise or code for a fusion protein comprising a segment of a Notch ligand extracellular domain and an immunoglobulin Fc segment.

Suitably the modulator of the Notch signalling pathway may comprise a fusion protein comprising a segment of a Notch ligand extracellular domain and an immunoglobulin Fc segment (e.g. IgG1 Fc or IgG4 Fc) or a polynucleotide coding for such a fusion protein. Suitable such fusion proteins are described, for example in Example 2 of WO 98/20142. IgG fusion proteins may be prepared as well known in the art, for example, as described in U.S. Pat. No. 5,428,130 (Genentech).

Suitably the modulator of the Notch signalling pathway comprises or codes for a protein or polypeptide comprising a Notch ligand DSL or EGF domain or a fragment, derivative, homologue, analogue or allelic variant thereof.

Preferably the modulator of the Notch signalling pathway comprises or codes for a Notch ligand DSL domain and at least one EGF-like domain, suitably at least 2, suitably at least 3, for example at least 3 to 16 or more EGF-like domains. Suitably the DSL and EGF sequences are or correspond to mammalian sequences. Preferred sequences include human sequences.

Alternatively or in addition the modulator of the Notch signalling pathway may comprise a Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

Suitably the modulator of the Notch signalling pathway comprises Delta or a fragment, derivative, homologue, analogue or allelic variant thereof or a polynucleotide encoding Delta or a fragment, derivative, homologue, analogue or allelic variant thereof.

Alternatively or in addition the modulator of the Notch signalling pathway may comprise Serrate/Jagged or a fragment, derivative, homologue, analogue or allelic variant thereof or a polynucleotide encoding Serrate/Jagged or a fragment, derivative, homologue, analogue or allelic variant thereof.

Alternatively or in addition the modulator of the Notch signalling pathway may comprise Notch or a fragment, derivative, homologue, analogue or allelic variant thereof or a polynucleotide encoding Notch or a fragment, derivative, homologue, analogue or allelic variant thereof.

Alternatively or in addition the modulator of the Notch signalling pathway may comprise a dominant negative version of a Notch signalling repressor, or a polynucleotide which codes for a dominant negative version of a Notch signalling repressor.

Alternatively or in addition the modulator of the Notch signalling pathway may comprise a polypeptide capable of upregulating the expression or activity of a Notch ligand or a downstream component of the Notch signalling pathway, or a polynucleotide which codes for such a polypeptide.

Suitably the modulator of the Notch signalling pathway may comprise an antibody, antibody fragment or antibody derivative or a polynucleotide which codes for an antibody, antibody fragment or antibody derivative.

Suitably the modulator of Notch signalling may be administered in a multimerised form. For example, in one embodiment the modulator of Notch signalling may be bound to a membrane or support. Suitably a plurality or multiplicity of modulators (for example at least 5) will be bound to the membrane or support. Such a membrane or support can be selected from those known in the art. In a preferred embodiment, the support is a particulate support matrix. In an even more preferred embodiment, the support is a bead. The bead may be, for example, a magnetic bead (e.g. as available under the trade name “Dynal”) or a polymeric bead such as a Sepharose bead.

Suitable the active agents described are administered in simultaneous, separate, or sequential combination with an antigen or antigenic determinant (such as an allergen, transplant antigen or autoantigen) or a polynucleotide coding for an antigen or antigenic determinant such that the immune response to said antigen or antigenic determinant may be modulated, preferably reduced.

In one preferred embodiment the invention employs a combination of a rapamycin or rapamycin derivative; and an activator of the Notch receptor, suitably being an activator which comprises or codes for a Notch ligand DSL domain and at least one EGF-like domain, suitably at least 1 to 20, suitably at least 2 to 16, for example at least 2 to 10 EGF-like domains; and optionally an antigen or antigenic determinant or a polynucleotide coding for an antigen or antigenic determinant.

In an alternative embodiment the invention employs a combination of a cyclosporin or cyclosporin derivative; and an activator of the Notch receptor, suitably being an activator which comprises or codes for a Notch ligand DSL domain and at least one EGF-like domain, suitably at least 1 to 20, suitably at least 2 to 16, for example at least 2 to 10 EGF-like domains; and optionally an antigen or antigenic determinant or a polynucleotide coding for an antigen or antigenic determinant.

In an alternative embodiment the invention employs a combination of FK506 or an FK506 derivative; and an activator of the Notch receptor, suitably being an activator which comprises or codes for a Notch ligand DSL domain and at least one EGF-like domain, suitably at least 1 to 20, suitably at least 2 to 16, for example at least 2 to 10 EGF-like domains; and optionally an antigen or antigenic determinant or a polynucleotide coding for an antigen or antigenic determinant.

According to a further aspect of the invention the immunophilin-binding immunosuppressive agent comprises a combination of a rapamycin or rapamycin derivative and a cyclosporin or a cyclosporin derivative.

According to a further embodiment the invention employs a combination of a rapamycin or rapamycin derivative; a cyclosporin or cyclosporin derivative; and an activator of the Notch receptor, suitably being an activator which comprises or codes for a Notch ligand DSL domain and at least one EGF-like domain, suitably at least 1 to 20, suitably at least 2 to 16, for example at least 2 to 10 EGF-like domains; and optionally an antigen or antigenic determinant or a polynucleotide coding for an antigen or antigenic determinant.

According to a further embodiment the invention employs a combination of a rapamycin or rapamycin derivative; FK506 or an FK506 derivative; and an activator of the Notch receptor, suitably being an activator which comprises or codes for a Notch ligand DSL domain and at least one EGF-like domain, suitably at least 1 to 20, suitably at least 2 to 16, for example at least 2 to 10 EGF-like domains; and optionally an antigen or antigenic determinant or a polynucleotide coding for an antigen or antigenic determinant.

In a further preferred embodiment the invention employs a combination of immunologically active steroid; and an activator of the Notch receptor, suitably being an activator which comprises or codes for a Notch ligand DSL domain and at least one EGF-like domain, suitably at least 1 to 20, suitably at least 2 to 16, for example at least 2 to 10 EGF-like domains; and optionally an antigen or antigenic determinant or a polynucleotide coding for an antigen or antigenic determinant.

Preferably the modulation of the immune system comprises modulation of T cell activity.

In one embodiment the modulation of the immune system comprises reduction of T cell activity. For example, the modulation of the immune system may comprise reduction of effector T-cell activity, for example reduction of helper (TH) and/or cytotoxic (TC) T-cell activity. Suitably the modulation of the immune system may comprise reduction of a Th1 or Th2 immune response.

Alternatively or in addition, the modulation of the immune system provides an increase of regulatory T-cell (T reg) activity, such as an increase of Tr1 or Th3 regulator T-cell activity.

Suitably the modulation of the immune system comprises generation of regulatory T cells (Tregs) and/or enhancement of Treg activity.

Suitably the modulation of the immune system comprises treatment of asthma, allergy, graft rejection, graft-versus-host disease or autoimmune disease.

Suitably the combinations of the invention may be used for modulation of expression of a cytokine selected from IL-10, IL-5, IL-4, IL-2, TNF-alpha, IFN-gamma or IL-13.

Suitably the combinations of the invention may be used for the manufacture of a medicament for increase of IL-10 expression.

Suitably the combinations of the invention may be used for the manufacture of a medicament for decrease of expression of a cytokine selected from IL-2, IL-4, IL-5, TNF-alpha, IFN-gamma or IL-13.

Suitably the combinations of the invention may be used for the manufacture of a medicament for generating an immune modulatory cytokine profile with increased IL-10 expression and reduced IL-4 or IL-5 expression.

Suitably the combinations of the invention may be used for the manufacture of a medicament for generating an immune modulatory cytokine profile with increased IL-10 expression and reduced IL-2, IFN-gamma, 1′-4, IL-5, IL-13 and TNF-alpha expression.

Thus the invention further provides a method for modulation of expression of a cytokine selected from IL-10, IL-5, IL-4, IL-2, TNF-alpha, IFN-gamma or IL-13 by administering a combination of active agents according to the present invention.

There is further provided a method for increase of IL-10 or IL-4 expression by administering a combination of active agents according to the present invention.

There is further provided a method for decrease of expression of a cytokine selected from IL-2, IL-5, TNF-alpha, IFN-gamma or IL-13 by administering a combination of active agents according to the present invention.

There is further provided a method for generating an immune modulatory cytokine profile with increased IL-10 expression and reduced IL-5 expression by administering a combination of active agents according to the present invention.

There is further provided a method for generating an immune modulatory cytokine profile with increased IL-10 expression and reduced IL-2, IFN-gamma, IL-5, IL-13 and TNF-alpha expression by administering a combination of active agents according to the present invention.

Suitably cytokine expression may be modified in leukocytes, fibroblasts or epithelial cells, preferably in dendritic cells, lymphocytes or macrophages, or their progenitors or tissue-specific derivatives.

According to a further aspect of the invention there is provided a method for producing a lymphocyte or antigen presenting cell (APC) capable of promoting tolerance which method comprises incubating a lymphocyte or APC obtained from a human or animal patient with (i) a modulator of the Notch signalling pathway and (ii) an immunosuppressive agent as described herein.

Suitably the method comprises incubating a lymphocyte or APC obtained from a human or animal patient with an APC in the presence of (i) a modulator of the Notch signalling pathway and (ii) an immunosuppressive agent as described herein.

According to a further aspect of the invention there is provided a method for producing an APC capable of inducing tolerance in a T cell which method comprises contacting an APC with (i) a modulator of the Notch signalling pathway and (ii) an immunosuppressive agent as described herein.

According to a further aspect of the invention there is provided a method for producing a lymphocyte or APC capable of promoting tolerance which method comprises incubating a lymphocyte or APC obtained from a human or animal patient with a lymphocyte or APC produced as described above.

Suitably in such methods the lymphocyte or APC may be incubated either in vivo or ex-vivo.

Suitably an antigen or antigenic determinant (or polynucleotide coding for an antigen or antigenic determinant) may also be administered as part of the methods, uses and products of the invention. The antigen or antigenic determinant (or polynucleotide coding for an antigen or antigenic determinant) may be administered in simultaneous, separate or sequential combination with the modulator of Notch signalling and immunosuppressive agent, so as to modulate, preferably reduce an immune response to the antigen or antigenic determinant.

In one embodiment the antigen or antigenic determinant may be an autoantigen or antigenic determinant thereof or a polynucleotide coding for an autoantigen or antigenic determinant thereof.

In another such embodiment the antigen or antigenic determinant may be an allergen or antigenic determinant thereof or a polynucleotide coding for an allergen or antigenic determinant thereof.

In another such embodiment the antigen or antigenic determinant may be a transplant antigen or antigenic determinant thereof or a polynucleotide coding for a transplant antigen or antigenic determinant thereof.

Suitably a modulator of Notch signalling for use in the present invention may comprise a protein or polypeptide comprising a Notch ligand DSL domain; 1-5 (and preferably not more than 5) Notch ligand EGF domains; optionally all or part of a Notch ligand N-terminal domain; and optionally one or more heterologous amino acid sequences; or a polynucleotide coding therefor.

Suitably a modulator of Notch signalling may comprise a protein or polypeptide comprising a Notch ligand DSL domain; 2-4 (and preferably not more than 4) Notch ligand EGF domains; optionally all or part of a Notch ligand N-terminal domain; and optionally one or more heterologous amino acid sequences; or a polynucleotide coding therefor.

Suitably one or more of the modulators of Notch signalling may comprise a protein or polypeptide comprising a Notch ligand DSL domain; 2-3 (and preferably not more than 3) Notch ligand EGF domains; optionally all or part of a Notch ligand N-terminal domain; and optionally one or more heterologous amino acid sequences; or a polynucleotide coding therefor.

Suitably the protein or polypeptide may have at least 50%, preferably at least 70%, preferably at least 90%, for example at least 95% amino acid sequence similarity (or preferably sequence identity) to the following sequence along the entire length of the latter:

| | |
|---|---|
| MGSRCALALAVLSALLCQVWSSGVFELKLQEFVNKKGLLGNRNCCRGGAGPPPCACRTF | (SEQ ID NO:24) |
| FRVCLKHYQASVSPEPPCTYGSAVTPVLGVDSFSLPDGGGADSAFSNPIRFPFGFTWPG | |
| TFSLIIEALHTDSPDDLATENPERLISRLATQRHLTVGEEWSQDLHSSGRTDLKYSYRF | |
| VCDEHYYGEGCSVFCRPRDDAFGHFTCGERGEKVCNPGWKGPYCTEPICLPGCDEQHGF | |
| CDKPGECKCRVGWQGRYCDECIRYPGCLHGTCQQPWQCNCQEGWGGLFCNQDLNYCTHH | |
| KPCKNGATCTNTGQGSYTCSCRPGYTGATCELGIDEC | |

### BRIEF DESCRIPTION OF THE DRAWINGS

Various preferred features and embodiments of the present invention will now be described in more detail by way of non-limiting example and with reference to the accompanying Figures, in which:

FIG. 1 shows a schematic representation of the Notch signalling pathway;

FIG. 2 shows schematic representations of the Notch ligands Jagged and Delta;

FIG. 3 shows aligned amino acid sequences of DSL domains from various Drosophila and mammalian Notch ligands; (SEQ ID NOs:1-16) FIGS. 4A-4C show the amino acid sequences of human Delta-1 (4A; SEQ ID NO:17), Delta-3 (4B; SEQ ID NO:18) and Delta-4 (4C; SEQ ID NO:19);

FIGS. 5A and 5B show the amino acid sequences of human Jagged-1 (SEQ ID NO:20) and Jagged-2 (SEQ ID NO:21), respectively;

FIG. 6 shows a coding (polynucleotide) sequence of human Delta-1 (SEQ ID NO:22);

FIGS. 7A and 7B shows a coding (polynucleotide) sequence of human Jagged-1 (SEQ ID NO:23);

FIG. 8 shows schematic representations of various Notch ligand domain/IgFc domain fusion proteins which may be used in the present invention;

FIG. 9 shows the results of Example 2;

FIG. 10 shows the results of Example 3;

FIG. 11 shows the results of Example 4; and

FIGS. 12 and 13 show the results of Example 5.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; and J. E. Coligan, A. M. Kruisbeek, D. H. Margulies, E. M. Shevach and W. Strober (1992 and periodic supplements; Current Protocols in Immunology, John Wiley & Sons, New York, N.Y.). Each of these general texts is herein incorporated by reference.

For the avoidance of doubt, Drosophila and vertebrate names are used interchangeably and all homologues are included within the scope of the invention.

Immunophilin-Binding Immunosuppressive Agent

The term “immunophilin-binding immunosuppressive agent” as used herein means an agent which binds to an immunophilin such as, for example, cyclophilin, an FK506-binding protein (FKBP), such as FKBP12 and FKBP50, mTOR (“Target Of Rapamycin”), RAPTOR or the like, so as to cause suppression of an immune response. Many of these immunophilins interact with calmodulin/calcineurin.

(a) Agents which Inhibit Transition of T Cells from the G1 to the GS Stages of the Cell Cycle

In one embodiment, the immunophilin-binding immunosuppressive agent may be an agent which by binding to an immunophilin inhibits transition of T cells from the G1 to the GS stages of the cell cycle, such as a rapamycin or rapamycin derivative.

The original (“native”) rapamycin (also known as sirolimus) is a macrolide compound isolated from the bacteria strain Streptomyces hygroscopicus having the following structure:

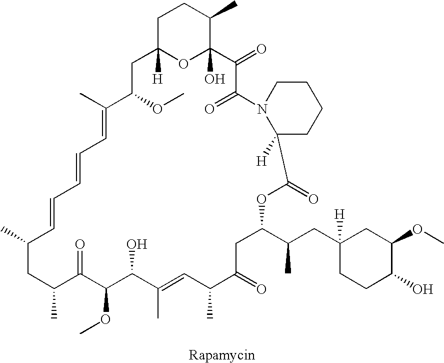

A large number of derivatives of rapamycin are known, which have the same type of activity, and which may also be used in accordance with the present invention.

Thus, the term “a rapamycin” as used herein includes esters, ethers, oximes, hydrazones, and hydroxylamines of native rapamycin, as well as rapamycins in which functional groups on the rapamycin nucleus have been modified, for example through reduction or oxidation (also termed “compounds of the rapamycin class”).

For example, as discussed in U.S. Pat. No. 6,399,625 (Wyeth) 42- and/or 31-esters and ethers of rapamycin are disclosed in the following patents, which are all hereby incorporated by reference: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 5,51,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers are disclosed in the patents listed above. The preparation of the corresponding esters and ethers of 1-oxorapamycin can be accomplished using the methodology described in these patents, starting with 1-oxorapamycin.

Preferred 27-esters and ethers of rapamycin are disclosed in U.S. Pat. No. 5,256,790, which is hereby incorporated by reference. The preparation of these esters and ethers are disclosed in the patents listed above. The preparation of the corresponding esters and ethers of 1-oxorapamycin can be accomplished using the methodology described in these patents, starting with 1-oxorapamycin.

Preferred oximes, hydrazones, and hydroxylamines of rapamycin are disclosed in U.S. Pat. Nos. 5,373,014, 5,378,836, 5,023,264, and 5,563,145, which are hereby incorporated by reference. The preparation of these oximes, hydrazones, and hydroxylamines are disclosed in the above listed patents. The preparation of 42-oxorapamycin is disclosed in U.S. Pat. No. 5,023,263, which is hereby incorporated by reference. The preparation of the corresponding oximes, hydrazones, and hydroxylamines of 1-oxorapamycin can be accomplished using the methodology described in these patents, starting with 1-oxorapamycin.

Particularly preferred 1-oxorapamycins include 1-oxorapamycin, 1-oxorapamycin 42-ester with 3-hydroxy-2-(hydroxymethyl)-2-methylpropionic acid [see U.S. Pat. No. 5,362,718 for the preparation of rapamycin 42-ester with 3-hydroxy-2-(hydroxymethyl)-2-methylpropionic acid], and 42-O-(2-hydroxy)ethyl 1-oxorapamycin [see U.S. Pat. No. 5,665,772 for the preparation of 42-O-(2-hydroxy)ethyl rapamycin; also known as “SDZ-RAD”].

Some further examples of rapamycin derivatives include, for example:
40-O-Benzyl-rapamycin
40-O-(4′-Hydroxymethyl)benzyl-rapamycin
40-O-[4′-(1,2-Dihydroxyethyl)]benzyl-rapamycin
40-O-Allyl-rapamycin
40-O-[3′-(2,2-Dimethyl-1,3-dioxolan-4(S)-yl]-prop-2′-en-1′-yl)-rapamycin (2′E, 4′S)-40-O-(4′,5′-Dihydroxypent-2′-en-1′-yl)-rapamycin
40-O-(2-Hydroxy)ethoxycarbonylmethyl-rapamycin
40-O-(2-Hydroxy)ethyl-rapamycin
40-O-(3-Hydroxy)propyl-rapamycin
40-O-(6-Hydroxy)hexyl-rapamycin
40-O-[2-(2-Hydroxy)ethoxy]ethyl-rapamycin
40-O-[(3S)-2,2-Dimethyldioxolan-3-yl]methyl-rapamycin
40-O-[(2S)-2,3-Dihydroxyprop-1-yl]-rapamycin
40-O-(2-Acetoxy)ethyl-raparnycin
40-O-(2-Nicotinoyloxy)ethyl-rapamycin
40-O-[2-(N-Morpholino)acetoxy]ethyl-rapamycin
40-O-(2-N-Imidazolylacetoxy)ethyl-rapamycin
40-O-[2-(N-Methyl-N′-piperazinyl)acetoxy]ethyl-rapamycin
39-O-Desmethyl-39,40-O,O-ethylene-rapamycin (26R)-26-Dihydro40-O-(2-hydroxy)ethyl-rapamycin
28-O-Methyl-rapamycin
40-O-(2-Aminoethyl)-rapamycin
40-O-(2-Acetaminoethyl)-rapamycin
40-O-(2-Nicotinamidoethyl)-rapamycin
40-O-(2-(N-Methyl-imidazo-2′-ylcarboxamido)ethyl)-rapamycin
40-O-(2-Ethoxycarbonylaminoethyl)-rapamycin
40-O-(2-Tolylsulfonamidoethyl)-rapamycin
40-O-[2-(4′,5′-Dicarboethoxy-1′,2′,3′-triazol-1′-yl)-ethyl]-rapamycin
40-O-(2-hydroxy)ethyl-rapamycin.
   (b) Agents which Inhibit Transition of T Cells from the G0 to the G1 Stages of the Cell Cycle

Alternatively or in addition, the immunophilin-binding immunosuppressive agent may be an agent which by binding to an immunophilin inhibits transition of T cells from the G0 to the G1 stages of the cell cycle, for example an agent which inhibits transcription of cytokines such as IL-2, for example a cyclosporin (e.g. cyclosporin A) or FK506 (tacrolimus) or a derivative thereof.

Cyclosporin A (CysA) is a cyclic undecapeptide which is obtained from a fungus (CAS No.59865-13-3; U.S. Pat. No. 3,737,433) and has the following structure:

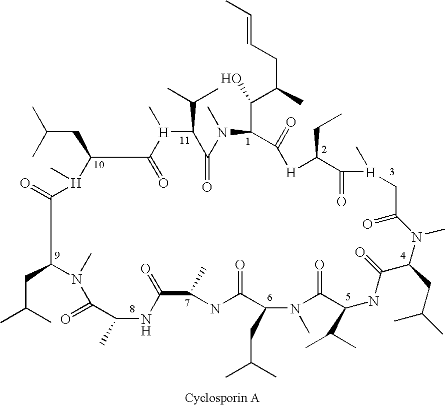

The term “a cyclosporin” as used herein means any of the group of cyclic oligopeptides with immunosuppressant activity and includes known cyclosporin A, cyclosporin B, cyclosporin C, cyclosporin D, cyclosporin E, cyclosporin F, cyclosporine G, cyclosporine H and cyclosporine I (also termed “compounds of the cyclosporin class”). In particular, this term includes cyclosporin A, also known as ciclosporine. Synthetically produced, naturally-derived or purified and recombinantly produced moieties are included within the definition, as are analogs, derivatives and pharmaceutically acceptable salts of any of these. The term also includes cyclosporins which have D-amino acids, modified, derivatized or non-naturally occurring amino acids in the D- or L-configuration and/or peptomimetic or prodrug units as part of their structure. Cyclosporin A is generally preferred.

Alternatively or in addition, the immunophilin-binding immunosuppressive agent may be an immunosuppressive ascomycin or ascomycin derivative (also termed “compounds of the ascomycin class”). Ascomycins, of which FK-506 and ascomycin are the best known, form a class of lactam macrolides, many of which have potent immunosuppressive and anti-inflammatory activity. FK506 is a lactam macrolide produced by Streptomyces tsukubaensis. Ascomycin is described e.g. in U.S. Pat. No. 3,244,592. Ascomycin; FK506, other naturally occurring macrolides having a similar biological activity and their derivatives, e.g. synthetic analogues and derivatives are termed collectively “Ascomycins”. Examples of synthetic analogues or derivatives are e.g. halogenated ascomycins, e.g. 33-epi-chloro-33-desoxy-ascomycin such as disclosed in EP-A-427,680, tetrahydropyran derivatives, e.g. as disclosed in EP-A-626,385

FK506 (also known as tacrolimus) is a macrolide having the following structure:

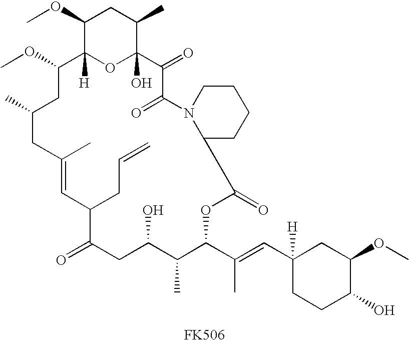

FK506 is a macrolide antibiotic produced by Streptomyces tsukubaensis No 9993. Methods of preparing FK506 are described in EP 184162.

A large number of derivatives of FK506, which retain the basic structure and at least one of the biological properties (for example immunological properties) of FK506, are known. Examples of such compounds are described, for example, in EP 184162, EP 315978, EP 323042, EP 423714, EP 427680, EP 465426, EP 474126, WO 91/13889, WO 91/19495, EP 484936, EP 532088, EP 532089, EP 569337, EP 626385 and WO 93/5059.

A preferred compound of the FK 506 class is disclosed in EP 427 680, e.g. Example 66a (also called 33-epi-chloro-33-desoxyascomycin). Other preferred compounds of the FK 506 class are disclosed in EP 465 426, EP 569 337, and in EP 626 385, for example the compound of Example 6d in EP 569 337, or the compound of Example 8 of EP 626385.

Immunologically Active Steroid

The term “immunologically active steroid” as used herein includes steroids which are capable of modifying immune responses. Preferably the immunologically active steroid activates the glucocorticoid (GC) receptor. Suitably the steroid modifies T cell, B cell or antigen presenting cell (APC) activity. Preferably the steroid reduces T cell, B cell or antigen presenting cell (APC) activity.

The term “immunologically active steroid” includes naturally occurring immunologically active steroids and their derivatives as well as synthetic or semi-synthetic steroid analogues having steroid-like immunological activity. Preferably the steroid is a corticosteroid or glucocorticoid. For example, many such steroids have a core fused ring structure based on cyclopentanophenanthrene. Examples of specific natural and synthetic steroids include, but are not limited to: aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol or triamcinolone, and their respective pharmaceutically acceptable salts or derivatives. It will be appreciated that combinations of such steroids may also be used in the invention.

As discussed in U.S. Published Patent Application No 20002009037 (DNAX) glucocorticoids (GC) are potent antiinflammatory and immunosuppressive agents that are widely used in the treatment of inflammatory disorders, such as autoimmune and allergic diseases (Wilkens and de Rijk (1997) Immunol. Today 18:418-424; Schleimer, et al. (ed. 1997) Inhaled Glucocorticoids in Asthma: Mechanisms & Clinical Actions Dekker. GC have been shown to have an inhibitory effect on both T cells and APC, at the level of proliferation as well as cytokine production, with down-regulation of IFN-g, IL-4, and IL-5 under some conditions, but upregulation of IL-4 under other conditions. See Blofta, et al. (1997) J. Immunol. 158:5589-5595; Ramirez, et al. (1996) J. Immunol. 156:2406-2412; and Daynes and Araneo (1989) Eur. J. Immunol. 19:2319-2325. This may result from indirect effects, e.g., GC down-regulate the production of IL-12 by APC and thus IFN-g production by T cells (Blotta, et al. (1997) J. Immunol. 158:5589-5595; Vieira, et al. (1998) J. Immunol. 161:5245-5251; and Visser, et al. (1998) Blood 91:4255-4264), and thus in some cases may indirectly upregulate the production of IL-4 (Blotta, et al. (1997) J. Immunol. 158:5589-5595) and or IL-5 (Vieira, et al. (1998) J. Immunol. 161:5245-5251) and/or IL-10 (Vieira, et al. (1998) J. Immunol. 161:5245-5251; and Visser, et al. (1998) Blood 91:4255-4264) in cultures contain APC, antigen, and T cells. Recently, it has been shown that GC drive human CD8+ T cell differentiation towards a stable phenotype with high IL-10 and reduced IFN-g, IL-4, IL-5 and IL-13 production. See Richards and Hawrylowicz (2000) Eur. J. Immunol. 30:2344-2354.

Glucocorticoids bind the cytosolic GC receptor (GR), which then translocates to the nucleus and inhibits the transcriptional activation of target genes. See review of Karin, in Schleimer, et al. (ed. 1997) Inhaled Glucocorticoids in Asthma: Mechanisms & Clinical Actions Dekker; and Karin (1998) Cell 93:487-490. Glucocorticoids mediate transcriptional repression through: 1) interfering with the function of transacting factors, such as AP-1 and NF-κB (De Bosscher, et al. (1997) 94:13504-13509), via protein-protein interactions; and inhibition of NFAT binding to cytokine gene promoters (Chen, et al. (2000) J. Immunol. 164:825-832); 2) direct DNA binding to poorly conserved negative GC responsive elements (GRE); or 3) inducing the expression of inhibitory factors such as IkBa (reviewed in Karin in Schleimer, et al. (eds. 1997) Inhaled Glucocorticoids in Asthma: Mechanisms & Clinical Actions Dekker). Furthermore, GR represses TGF-b transcriptional activation of the plasminogen activator PAI-1 gene in a ligand-dependent manner, by both Smad3 and Smad4 C-terminal activation domains. See Song, et al. (1999) Proc. Nat'l Acad. Sci. USA 96:11776-11781.

Modulators of Notch Signalling

The term “modulation of the Notch signalling pathway” as used herein refers to a change or alteration in the biological activity of the Notch signalling pathway or a target signalling pathway thereof. The term “modulator of the Notch signalling pathway” (or “modulator of Notch signalling”) may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, and preferably the term “modulator of the Notch signalling pathway” may refer to agonists (or partial agonists) of Notch signalling, i.e. compounds which stimulate or upregulate, at least to some extent, the normal biological activity of the Notch signalling pathway. Conveniently such compounds may be referred to as upregulators or agonists. Preferably the modulator of Notch signalling used is an agonist of Notch signalling, and preferably an agonist of the Notch receptor (e.g. an agonist of the Notch1, Notch2, Notch3 and/or Notch4 receptor, preferably being a human Notch receptor). Preferably such an agonist (“activator of Notch”) binds to and activates a Notch receptor, preferably including human Notch recpetors such as human Notch1, Notch2, Notch3 and/or Notch4. Binding to and/or activation of a Notch receptor may be assessed by a variety of techniques known in the art including in vitro binding assays and activity assays for example as described herein.

For example, whether any particular agent activates Notch signalling (e.g. is an activator of Notch or a Notch agonist) may be readily determined by use of any suitable assay, for example by use of a CBF-1 reporter assay of the type described in WO03/012441 in the name of Lorantis Ltd (e.g. see Examples 8 and 9 therein). Conversely, antagonist activity may be readily determined for example by monitoring any effect of the agent in reducing signalling by known Notch signalling agonists for example, as described in WO03/012441 or WO 03/041735 in the name of Lorantis Ltd (e.g. see Examples 10,11 and 12) (i.e. in a so-called “antagonist” assay).

The active agent of the present invention may for example be an organic compound or other chemical. In one embodiment, a modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In one preferred embodiment, the modulator will be an amino acid sequence or a chemical derivative thereof. In another preferred embodiment, the modulator will be a nucleotide sequence—which may be a sense sequence or an anti-sense sequence. The modulator may also be an antibody.

The term “antibody” includes intact molecules as well as fragments thereof, such as Fab, F(ab′)2, Fv and scFv which are capable of binding the epitopic determinant. These antibody fragments retain some ability to selectively bind with its antigen or receptor and include, for example:
(i) Fab, the fragment which contains a monovalent antigen-binding fragment of an antibody molecule can be produced by digestion of whole antibody with the enzyme papain to yield an intact light chain and a portion of one heavy chain;
(ii) Fab′, the fragment of an antibody molecule can be obtained by treating whole antibody with pepsin, followed by reduction, to yield an intact light chain and a portion of the heavy chain; two Fab′ fragments are obtained per antibody molecule;
(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;
(iv) scFv, including a genetically engineered fragment containing the variable region of a heavy and a light chain as a fused single chain molecule.

General methods of making these fragments are known in the art. (See for example, Harlow and Lane, Antibodies: A Laboratory Manual, Cold Spring Harbor Laboratory, New York (1988), which is incorporated herein by reference).

For example, antibodies against Notch and Notch ligands are described in U.S. Pat. Nos. 5,648,464, 5,849,869 and 6,004,924 (Yale University/Imperial Cancer Technology), the texts of which are herein incorporated by reference.

Antibodies generated against the Notch receptor are also described in WO 0020576 (the text of which is also incorporated herein by reference). For example, this document discloses generation of antibodies against the human Notch-1 EGF-like repeats 11 and 12. For example, in particular embodiments, WO 0020576 discloses a monoclonal antibody secreted by a hybridoma designated A6 having the ATCC Accession No. HB12654, a monoclonal antibody secreted by a hybridoma designated C11 having the ATCC Accession No. HB12656 and a monoclonal antibody secreted by a hybridoma designated F3 having the ATCC Accession No. HB12655.

An anti-human-Jagged1 antibody is available from R & D Systems, Inc, reference MAB12771 (Clone 188323).

Preferably a modulator of Notch signalling will be in a multimerised form, and may preferably comprise a construct comprising at least 3, preferably at least 5, preferably at least 10, at least 30, or at least 50 or 100 or more modulators of Notch signalling.

For example, modulators of Notch signalling in the form of Notch ligand proteins/polypeptides coupled to particulate supports such as beads are described in WO 03/011317 (Lorantis) and in Lorantis' co-pending PCT application PCT/GB2003/001525 (filed on 4 Apr. 2003), the texts of which are hereby incorporated by reference (e.g. see in particular Examples 17, 18, 19 of PCT/GB2003/001525).

Modulators of Notch signalling in the form of Notch ligand proteins/polypeptides coupled to polymer supports are described in Lorantis Ltd's co-pending PCT application PCT/GB2003/003285 (filed on 1 Aug. 2003 claiming priority from GB 0218068.5), the text of which is herein incorporated by reference (e.g. see in particular Example 5 therein disclosing a dextran conjugate).

In one form the agent for modulation of the Notch signalling pathway may be a protein for Notch signalling transduction.

By a protein which is for Notch signalling transduction is meant a molecule which participates in signalling through Notch receptors including activation of Notch, the downstream events of the Notch signalling pathway, transcriptional regulation of downstream target genes and other non-transcriptional downstream events (e.g. post-translational modification of existing proteins). More particularly, the protein may comprise a domain that allows activation of target genes of the Notch signalling pathway, or a polynucleotide sequence which codes therefor.

A very important component of the Notch signalling pathway is Notch receptor/Notch ligand interaction. Thus Notch signalling may involve changes in expression, nature, amount or activity of Notch ligands or receptors or their resulting cleavage products. In addition, Notch signalling may involve changes in expression, nature, amount or activity of Notch signalling pathway membrane proteins or G-proteins or Notch signalling pathway enzymes such as proteases, kinases (e.g. serine/threonine kinases), phosphatases, ligases (e.g. ubiquitin ligases) or glycosyltransferases. Alternatively the signalling may involve changes in expression, nature, amount or activity of DNA binding elements such as transcription factors.

In the present invention Notch signalling preferably means specific signalling, meaning that the signalling results substantially or at least predominantly from the Notch signalling pathway, and preferably from Notch/Notch ligand interaction, rather than any other significant interfering or competing cause, such as cytokine signalling. Preferably therefor the term “Notch signalling” as used herein excludes cytokine signalling. The Notch signalling pathway is described in more detail below.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Key targets for Notch-dependent transcriptional activation are genes of the Enhancer of split complex (E[spl]). Moreover these genes have been shown to be direct targets for binding by the Su(H) protein and to be transcriptionally activated in response to Notch signalling. By analogy with EBNA2, a viral coactivator protein that interacts with a mammalian Su(H) homologue CBF1 to convert it from a transcriptional repressor to a transcriptional activator, the Notch intracellular domain, perhaps in association with other proteins may combine with Su(H) to contribute an activation domain that allows Su(H) to activate the transcription of E(spl) as well as other target genes. It should also be noted that Su(H) is not required for all Notch-dependent decisions, indicating that Notch mediates some cell fate choices by associating with other DNA-binding transcription factors or be employing other mechanisms to transduce extracellular signals.

According to one aspect of the present invention the active agent may be Notch or a fragment thereof which retains the signalling transduction ability of Notch or an analogue of Notch which has the signalling transduction ability of Notch.

As used herein the term “analogue of Notch” includes variants thereof which retain the signalling transduction ability of Notch. By “analogue” we include a protein which has Notch signalling transduction ability, but generally has a different evolutionary origin to Notch. Analogues of Notch include proteins from the Epstein Barr virus (EBV), such as EBNA2, BARF0 or LMP2A.

By a protein which is for Notch signalling activation we mean a molecule which is capable of activating Notch, the Notch signalling pathway or any one or more of the components of the Notch signalling pathway.

In one embodiment, the active agent may be a Notch ligand, or a polynucleotide encoding a Notch ligand. Notch ligands of use in the present invention include endogenous Notch ligands which are typically capable of binding to a Notch receptor polypeptide present in the membrane of a variety of mammalian cells, for example hemapoietic stem cells.

The term “Notch ligand” as used herein means an agent capable of interacting with a Notch receptor to cause a biological effect. The term as used herein therefore includes naturally occurring protein ligands such as Delta and Serrate/Jagged as well as antibodies to the Notch receptor, peptidomimetics and small molecules which have corresponding biological effects to the natural ligands. Preferably the Notch ligand interacts with the Notch receptor by binding.

Particular examples of mammalian Notch ligands identified to date include the Delta family, for example Delta or Delta-like 1 (Genbank Accession No. AF003522-Homo sapiens), Delta-3 (Genbank Accession No. AF084576-Rattus norvegicus) and Delta-like 3 (Mus musculus) (Genbank Accession No. NM_{—}016941-Homo sapiens) and U.S. Pat. No. 6,121,045 (Millennium), Delta-4 (Genbank Accession Nos. AB043894 and AF 253468-Homo sapiens) and the Serrate family, for example Serrate-1 and Serrate-2 (WO97/01571, WO96/27610 and WO92/19734), Jagged-1 (Genbank Accession No. U73936-Homo sapiens) and Jagged-2 (Genbank Accession No. AF029778-Homo sapiens), and LAG-2. Homology between family members is extensive.

In a further embodiment, an activator may be a constitutively active Notch receptor or Notch intracellular domain, or a polynucleotide encoding such a receptor or intracellular domain.

In an alternative embodiment, an activator of Notch signalling will act downstream of the Notch receptor. Thus, for example, the activator of Notch signalling may be a constitutively active Deltex polypeptide or a polynucleotide encoding such a polypeptide. Other downstream components of the Notch signalling pathway of use in the present invention include the polypeptides involved in the Ras/MAPK cascade catalysed by Deltex, polypeptides involved in the proteolytic cleavage of Notch such as Presenilin and polypeptides involved in the transcriptional regulation of Notch target genes, preferably in a constitutively active form.

By polypeptide for Notch signalling activation is also meant any polypeptides expressed as a result of Notch activation and any polypeptides involved in the expression of such polypeptides, or polynucleotides coding for such polypeptides.

Preferably when the inhibitor is a receptor or a nucleic acid sequence encoding a receptor, the receptor is activated. Thus, for example, when the agent is a nucleic acid sequence, the receptor is preferably constitutively active when expressed.

Any one or more of appropriate targets—such as an amino acid sequence and/or nucleotide sequence—may be used for identifying a compound capable of modulating the Notch signalling pathway and/or a targeting molecule in any of a variety of drug screening techniques. The target employed in such a test may be free in solution, affixed to a solid support, borne on a cell surface, or located intracellularly.

Techniques for drug screening may be based on the method described in Geysen, European Patent No. 0138855, published on Sep. 13, 1984. In summary, large numbers of different small peptide candidate modulators or targeting molecules are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators and targeting molecules.

This invention also contemplates the use of competitive drug screening assays in which neutralising antibodies capable of binding a target specifically compete with a test compound for binding to a target.

Techniques are well known in the art for the screening and development of agents such as antibodies, peptidomimetics and small organic molecules which are capable of binding to and modulating components of the Notch signalling pathway. These include the use of phage display systems for expressing signalling proteins, and using a culture of transfected E. coli or other microorganism to produce the proteins for binding studies of potential binding compounds (see, for example, G. Cesarini, FEBS Letters, 307 (1):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

Polypeptides, Proteins and Amino Acid Sequences

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

“Peptide” usually refers to a short amino acid sequence that is 10 to 40 amino acids long, preferably 10 to 35 amino acids.

The amino acid sequence may be prepared and isolated from a suitable source, or it may be made synthetically or it may be prepared by use of recombinant DNA techniques.

Within the definitions of “proteins” useful in the present invention, the specific amino acid residues may be modified in such a manner that the protein in question retains at least one of its endogenous functions, such modified proteins are referred to as “variants”. A variant protein can be modified by addition, deletion and/or substitution of at least one amino acid present in the naturally-occurring protein.

Typically, amino acid substitutions may be made, for example from 1, 2 or 3 to 10 or 20 substitutions provided that the modified sequence retains the required target activity or ability to modulate Notch signalling. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the target or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| Symbol | 3-letter | Meaning | Codons |
|---|---|---|---|
| A | Ala | Alanine | GCT, GCC, GCA, GCG |
| B | Asp, | Aspartic, | GAT, GAC, AAT, AAC |
| | Asn | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, CTA, CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, AGA, AGG |
| S | Ser | Serine | TCT, TCC, TCA, TCG, AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, | Glutamic, | GAA, GAG, CAA, CAG |
| | Gln | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function. A peptide useful in the invention will at least have a target or signalling modulation capability. “Fragments” are also variants and the term typically refers to a selected region of the protein that is of interest in a binding assay and for which a binding partner is known or determinable. “Fragment” thus refers to an amino acid sequence that is a portion of a full-length polypeptide, for example between about 8 and about 1500 amino acids in length, typically between about 8 and about 745 amino acids in length, preferably about 8 to about 300, more preferably about 8 to about 200 amino acids, and even more preferably about 10 to about 50 or 100 amino acids in length. “Peptide” preferably refers to a short amino acid sequence that is 10 to 40 amino acids long, preferably 10 to 35 amino acids.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Variants of the nucleotide sequence may also be made. Such variants will preferably comprise codon optimised sequences. Codon optimisation is known in the art as a method of enhancing RNA stability and therefore gene expression. The redundancy of the genetic code means that several different codons may encode the same amino-acid. For example, leucine, arginine and serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms.

Proteins or polypeptides may be in the form of the “mature” protein or may be a part of a larger protein such as a fusion protein or precursor. For example, it is often advantageous to include an additional amino acid sequence which contains secretory or leader sequences or pro-sequences (such as a HIS oligomer, immunoglobulin Fc, glutathione S-transferase, FLAG etc) to aid in purification. Likewise such an additional sequence may sometimes be desirable to provide added stability during recombinant production. In such cases the additional sequence may be cleaved (e.g. chemically or enzymatically) to yield the final product. In some cases, however, the additional sequence may also confer a desirable pharmacological profile (as in the case of IgFc fusion proteins) in which case it may be preferred that the additional sequence is not removed so that it is present in the final product as administered.

Where the modulator of Notch signalling or antigen/antigenic determinant comprises a nucleotide sequence it may suitably be codon optimised for expression in mammalian cells. In a preferred embodiment, such sequences are optimised in their entirety.

Nucleic acids and Polynucleotides

In one embodiment the modulator of Notch signalling may be a polynucleotide, for example a polynucleotide coding for a Notch ligand such as Delta or Serrate or an active portion thereof. Suitably, for example, such a polynucleotide may code for a Notch ligand DSL domain and at least one EGF domain, preferably at least 3 EGF domains. Suitably the polynucleotide may also code for a Notch ligand transmembrane domain and preferably also a Notch ligand intracellular domain.

Such polynucleotides may for example be administered by conventional DNA delivery techniques, such as DNA vaccination etc, or injected or otherwise delivered for example with needleless systems. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

“Polynucleotide” refers to a polymeric form of nucleotides of at least 10 bases in length and up to 10,000 bases or more, either ribonucleotides or deoxyribonucleotides or a modified form of either type of nucleotide. The term includes single and double stranded forms of DNA and RNA and also derivatised versions such as protein nucleic acid (PNA).

These may be constructed using standard recombinant DNA methodologies. The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference may be made to Molecular Cloning by Sambrook et al. (Cold Spring Harbor, 1989) or similar standard reference books for exact details of the appropriate techniques.

Nucleic acid encoding the second region may likewise be provided in a similar vector system.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data are available or where it is desired to express a nucleic acid homologous or otherwise related to a known nucleic acid, exemplary nucleic acids can be characterised as those nucleotide sequences which hybridise to the nucleic acid sequences known in the art.

It will be understood by a skilled person that numerous different nucleotide sequences can encode the same protein used in the present invention as a result of the degeneracy of the genetic code. In addition, it is to be understood that skilled persons may, using routine techniques, make nucleotide substitutions that do not affect the protein encoded by the nucleotide sequence of the present invention to reflect the codon usage of any particular host organism in which the target protein or protein for Notch signalling modulation of the present invention is to be expressed.

In general, the terms “variant”, “homologue” or “derivative” in relation to the nucleotide sequence used in the present invention includes any substitution of, variation of, modification of, replacement of, deletion of or addition of one (or more) nucleic acid from or to the sequence providing the resultant nucleotide sequence codes for a modulator of Notch signalling and retains corresponding activity.

As indicated above, with respect to sequence homology, preferably there is at least 40%, preferably at least 70%, preferably at least 75%, more preferably at least 85%, more preferably at least 90% homology to the reference sequences. More preferably there is at least 95%, more preferably at least 98%, homology. Nucleotide homology comparisons may be conducted as described above. A preferred sequence comparison program is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the reference sequences, or any variant, fragment or derivative thereof, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction (PCR) technologies.

Nucleotide sequences useful in the invention capable of selectively hybridising to the nucleotide sequences presented herein, or to their complement, will be generally at least 75%, preferably at least 85 or 90% and more preferably at least 95% or 98% homologous to the corresponding nucleotide sequences presented herein over a region of at least 20, preferably at least 25 or 30, for instance at least 40, 60 or 100 or more contiguous nucleotides. Preferred nucleotide sequences of the invention will comprise regions homologous to the nucleotide sequence, preferably at least 80 or 90% and more preferably at least 95% homologous to the nucleotide sequence.

The term “selectively hybridizable” means that the nucleotide sequence used as a probe is used under conditions where a target nucleotide sequence of the invention is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃ Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences can be obtained in a number of ways. Variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the modulator of Notch signalling encoded by the nucleotide sequences.

The nucleotide sequences such as a DNA polynucleotides useful in the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector. For larger genes, portions may be cloned separately in this way and then ligated to form the complete sequence.

Protein and Polypeptide Expression

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transvection, microinjection, cationic lipid-mediated transfection, electroporation, transduction, scrape loading, ballistic introduction and infection. In will be appreciated that such methods can also be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; T-cell lines such as Jurkat cells; B-cell lines such as A20 cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Techniques for refolding protein may be employed to regenerate active conformation when the polypeptide is denatured during isolation and/or purification.

Polypeptides and Polynucleotides for Notch Signalling Transduction

Notch was first described in Drosophila as a transmembrane protein that functions as a receptor for two different ligands, Delta and Serrate. Vertebrates express multiple Notch receptors and ligands (discussed below). At least four Notch receptors (Notch-1, Notch-2, Notch-3 and Notch-4) have been identified to date in human cells (see for example GenBank Accession Nos. AF308602, AF308601 and U95299-Homo sapiens).

The Notch signalling pathway directs binary cell fate decisions in the embryo. Notch was first described in Drosophila as a transmembrane protein that functions as a receptor for two different ligands, Delta and Serrate. Vertebrates express multiple Notch receptors and ligands (discussed below).

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs in the Golgi apparatus and is mediated by a furin-like convertase.

Notch receptors are inserted into the membrane as heterodimeric molecules comprising an extracellular domain containing up to 36 epidermal growth factor (EGF)-like repeats [Notch 1/2=36, Notch 3=34 and Notch 4=29], 3 Cysteine Rich Repeats (Lin-Notch (L/N) repeats) and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and is involved in binding to a transcription factor, known as Suppressor of Hairless [Su(H)] in Drosophila and CBF1 in vertebrates (Tamura K, et al. (1995) Curr. Biol. 5:1416-1423 (Tamura)). The Notch ligands also display multiple EGF-like repeats in their extracellular domains together with a cysteine-rich DSL (Delta-Serrate Lag2) domain that is characteristic of all Notch ligands (Artavanis-Tsakomas et al. (1995) Science 268:225-232, Artavanis-Tsakomas et al. (1999) Science 284:770-776).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber et al. (1993) Genes Dev 7(10):1949-65 (Lieber)). Recent studies have indeed shown that Notch activation requires that the six cdc10/ankyrin repeats of the Notch intracellular domain reach the nucleus and participate in transcriptional activation. The site of proteolytic cleavage on the intracellular tail of Notch has been identified between gly1743 and val 744 (termed site 3, or S3) (Schroeter, E. H. et al. (1998) Nature 393 (6683):382-6 (Schroeter)). It is thought that the proteolytic cleavage step that releases the cdc10/ankyrin repeats for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl, G. et al. (1998) Cell 93 (4):649-60 (Struhl)). The NotchIC—CBF1 complexes then activate target genes, such as the bHLH proteins HES (hairy-enhancer of split like) 1 and 5 (Weinmaster G. (2000) Curr. Opin. Genet. Dev. 10:363-369 (Weinmaster)). This nuclear function of Notch has also been shown for the mammalian Notch homologue (Lu, F. M. et al. (1996) Proc Natl Acad Sci 93 (11):5663-7 (Lu)).

S3 processing occurs only in response to binding of Notch ligands Delta or Serrate/Jagged. The post-translational modification of the nascent Notch receptor in the Golgi (Munro S, Freeman M. (2000) Curr. Biol. 10:813-820 (Munro); Ju B J, et al. (2000) Nature 405:191-195 (Ju)) appears, at least in part, to control which of the two types of ligand is expressed on a cell surface. The Notch receptor is modified on its extracellular domain by Fringe, a glycosyl transferase enzyme that binds to the Lin/Notch motif. Fringe modifies Notch by adding O-linked fucose groups to the EGF-like repeats (Moloney D J, et al. (2000) Nature 406:369-375 (Moloney), Brucker K, et al. (2000) Nature 406:411-415 (Brucker)). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially bind Delta (Panin V M, et al. (1997) Nature 387:908-912 (Panin), Hicks C, et al. (2000) Nat. Cell. Biol. 2:515-520 (Hicks)). Although Drosophila has a single Fringe gene, vertebrates are known to express multiple genes (Radical, Manic and Lunatic Fringes) (Irvine K D (1999) Curr. Opin. Genet. Devel. 9:434-441 (Irvine)).

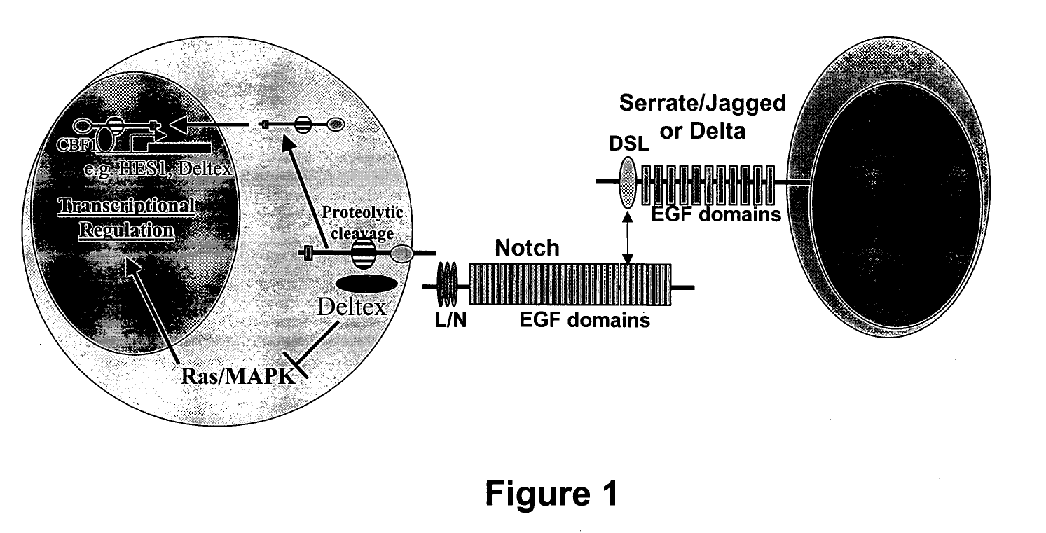

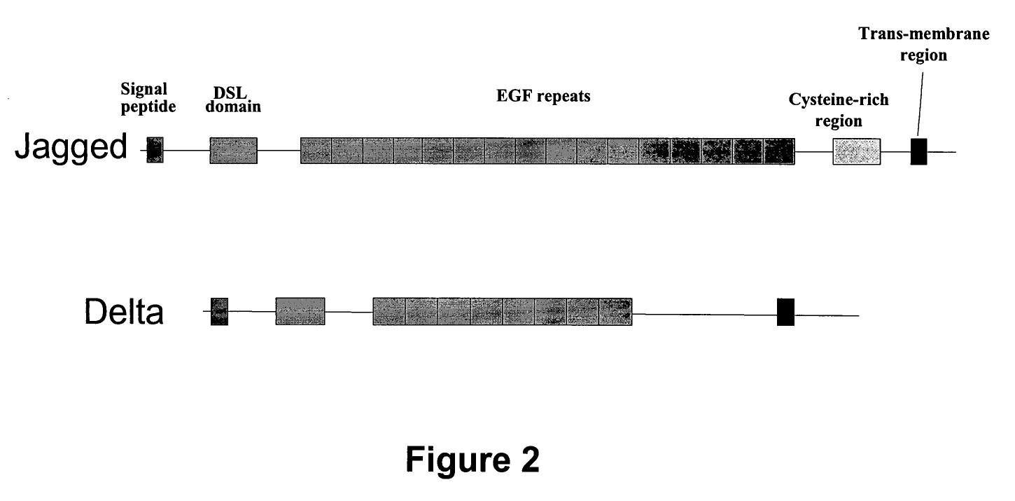

Signal transduction from the Notch receptor can occur via two different pathways (see e.g. FIG. 1). The better defined pathway involves proteolytic cleavage of the intracellular domain of Notch (Notch IC) that translocates to the nucleus and forms a transcriptional activator complex with the CSL family protein CBF1 (suppressor of Hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex. Unlike CBF1, Deltex does not move to the nucleus following Notch activation but instead can interact with Grb2 and modulate the Ras-JNK signalling pathway.

Target genes of the Notch signalling pathway include Deltex, genes of the Hes family (Hes-1 in particular), Enhancer of Split [E(spl)] complex genes, IL-10, CD-23, CD-4 and Dll-1.

Deltex, an intracellular docking protein, replaces Su(H) as it leaves its site of interaction with the intracellular tail of Notch. Deltex is a cytoplasmic protein containing a zinc-finger (Artavanis-Tsakomas et al. (1995) Science 268:225-232; Artavanis-Tsakomas et al. (1999) Science 284:770-776; Osborne B, Miele L. (1999) Immunity 11:653-663 (Osborne)). It interacts with the ankyrin repeats of the Notch intracellular domain. Studies indicate that Deltex promotes Notch pathway activation by interacting with Grb2 and modulating the Ras-JNK signalling pathway (Matsuno et al. (1995) Development 121 (8):2633-44; Matsuno K, et al. (1998) Nat. Genet. 19:74-78). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich et al. (1998) Mol. Cell. Biol. 18:2230-2239 (Ordentlich)). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi K. et al. (1994) J Biol Chem 269 (7):150-6 (Takebayashi)) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of Mus musculus Hes-1 can be found in GenBank Accession No. D16464.

The E(spl) gene complex [E(spl)-C] (Leimeister C. et al. (1999) Mech Dev 85 (1-2):173-7 (Leimeister)) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

Interleukin-10 (IL-10) was first characterised in the mouse as a factor produced by Th2 cells which was able to suppress cytokine production by Th1 cells. It was then shown that IL-10 was produced by many other cell types including macrophages, keratinocytes, B cells, Th0 and Th1 cells. It shows extensive homology with the Epstein-Barr bcrf1 gene which is now designated viral IL-10. Although a few immunostimulatory effects have been reported, it is mainly considered as an immunosuppressive cytokine. Inhibition of T cell responses by IL-10 is mainly mediated through a reduction of accessory functions of antigen presenting cells. IL-10 has notably been reported to suppress the production of numerous pro-inflammatory cytokines by macrophages and to inhibit co-stimulatory molecules and MHC class II expression. IL-10 also exerts anti-inflammatory effects on other myeloid cells such as neutrophils and eosinophils. On B cells, IL-10 influences isotype switching and proliferation. More recently, IL-10 was reported to play a role in the induction of regulatory T cells and as a possible mediator of their suppressive effect. Although it is not clear whether it is a direct downstream target of the Notch signalling pathway, its expression has been found to be strongly up-regulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. The sequence for CD-23 may be found in GenBank ref. No. GI1783344.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF 1 response elements and its expression is upregulated as a result of Notch activation. The sequence of CTLA4 can be found in GenBank Accession No. L15006.

Dlx-1 (distalless-1) (McGuinness T. Et al (1996) Genomics 35 (3):473-85 (McGuiness)) expression is downregulated as a result of Notch activation. Sequences for Dlx genes may be found in GenBank Accession Nos. U51000-3.

CD-4 expression is downregulated as a result of Notch activation. A sequence for the CD-4 antigen may be found in GenBank Accession No. XM006966.

As described above the Notch receptor family participates in cell-cell signalling events that influence T cell fate decisions. In this signalling NotchIC localises to the nucleus and functions as an activated receptor. Mammalian NotchIC interacts with the transcriptional repressor CBF1. It has been proposed that the NotchIC cdc10/ankyrin repeats are essential for this interaction. Hsieh et al (Hsieh et al. (1996) Molecular & Cell Biology 16 (3):952-959) suggests rather that the N-terminal 114 amino acid region of mouse NotchIC contains the CBF1 interactive domain. It is also proposed that NotchIC acts by targeting DNA-bound CBF1 within the nucleus and abolishing CBF1-mediated repression through masking of the repression domain. It is known that Epstein Barr virus (EBV) immortalizing protein EBNA” also utilises CBF1 tethering and masking of repression to upregulate expression of CBF1-repressed B-cell genes. Thus, mimicry of Notch signal transduction is involved in EBV-driven immortalization. Strobl et al (Strobl et al. (2000) J Virol 74 (4): 1727-35) similarly reports that “EBNA2 may hence be regarded as a functional equivalent of an activated Notch receptor”. Other EBV proteins which fall in this category include BARF0 (Kusano and Raab-Truab (2001) J Virol 75 (1):384-395 (Kusano and Raab-Traub)) and LMP2A.

Other genes involved in the Notch signaling pathway, such as Numb, Mastermind and Dsh, and all genes the expression of which is modulated by Notch activation, are included in the scope of this invention.

Polypeptides and Polynucleotides for Notch Signalling Activation

In a preferred embodiment the modulator of Notch signalling will be an agent for Notch signalling activation, preferably a Notch ligand or fragment, variant, derivative, homologue or mimetic thereof.

Examples of mammalian Notch ligands identified to date include the Delta family, for example Delta-1 (Genbank Accession No. AF003522-Homo sapiens), Delta-3 (Genbank Accession No. AF084576-Rattus norvegicus) and Delta-like 3 (Mus musculus), the Serrate family, for example Serrate-1 and Serrate-2 (WO97/01571, WO96/27610 and WO92/19734), Jagged-1 and Jagged-2 (Genbank Accession No. AF029778-Homo sapiens), and LAG-2. Homology between family members is extensive.

Further homologues of known mammalian Notch ligands may be identified using standard techniques. By a “homologue” it is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequence of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example http://www.ncbi.nlm.nih.gov and Ausubel et al., Current Protocols in Molecular Biology (1995), John Wiley & Sons, Inc.).

Notch ligands identified to date have a diagnostic DSL domain (D. Delta, S. Serrate, L. Lag2) comprising 20 to 22 amino acids at the amino terminus of the protein and up to 14 or more EGF-like repeats on the extracellular surface. It is therefore preferred that homologues of Notch ligands also comprise a DSL domain at the N-terminus and up to 14 or more EGF-like repeats on the extracellular surface.

In addition, suitable homologues will preferably be capable of binding to a Notch receptor. Binding may be assessed by a variety of techniques known in the art including in vitro binding assays and activation of the receptor (in the case of an agonist or partial agonist) may be determined for example by use of assays as described in the Examples hereto and in WO 03/012441 (Lorantis) the text of which is hereby incorporated herein by reference.

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Polypeptide substances may be purified from mammalian cells, obtained by recombinant expression in suitable host cells or obtained commercially. Alternatively, nucleic acid constructs encoding the polypeptides may be used. As a further example, overexpression of Notch or Notch ligand, such as Delta or Serrate, may be brought about by introduction of a nucleic acid construct capable of activating the endogenous gene, such as the Serrate or Delta gene. In particular, gene activation can be achieved by the use of homologous recombination to insert a heterologous promoter in place of the natural promoter, such as the Serrate or Delta promoter, in the genome of the target cell.

The activating molecule of the present invention may, in an alternative embodiment, be capable of modifying Notch-protein expression or presentation on the cell membrane or signalling pathways. Agents that enhance the presentation of a fully functional Notch-protein on the target cell surface include matrix metalloproteinases such as the product of the Kuzbanian gene of Drosophila (Dkuz) and other ADAMALYSIN gene family members.

Notch Ligand Domains

As discussed above, Notch ligands typically comprise a number of distinctive domains. Some predicted/potential domain locations for various naturally occurring human Notch ligands (based on amino acid numbering in the precursor proteins) are shown below:

| | | |
|---|---|---|
| Component | Amino acids | Proposed function/domain |

Human Delta 1

| | | |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| | | |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| | | |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| | | |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| | | |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence (SEQ ID NO:25):

Cys Xaa Xaa Xaa Xaa Xaa Xaa Xaa Xaa Cys Xaa Xaa Xaa Cys Xaa Xaa
Xaa Xaa Xaa Xaa Xaa Xaa Xaa Xaa Xaa Cys Xaa Xaa Xaa Xaa Xaa Xaa
Xaa Cys Xaa Xaa Xaa Xaa Xaa Xaa Xaa Xaa Cys

Preferably the DSL domain may include most or all of the following consensus amino acid sequence (SEQ ID NO:26):

Cys Xaa Xaa Xaa ARO ARO Xaa Xaa Xaa Cys Xaa Xaa Xaa Cys BAS NOP
BAS ACM ACM Xaa ARO NOP ARO Xaa Xaa Cys Xaa Xaa Xaa NOP Xaa Xaa
Xaa Cys Xaa Xaa NOP ARO Xaa NOP Xaa Xaa Cys

wherein:
ARO is an aromatic amino acid residue, such as tyrosine, phenylalanine, tryptophan or histidine;
NOP is a non-polar amino acid residue such as glycine, alanine, proline, leucine, isoleucine or valine;
BAS is a basic amino acid residue such as arginine or lysine; and
ACM is an acid or amide amino acid residue such as aspartic acid, glutamic acid, asparagine or glutamine.

Preferably the DSL domain may include most or all of the following consensus amino acid sequence (SEQ ID NO:27):

Cys Xaa Xaa Xaa Tyr Tyr Xaa Xaa Xaa Cys Xaa Xaa Xaa Cys Arg Pro
Arg Asx Asp Xaa Phe Gly His Xaa Xaa Cys Xaa Xaa Xaa Gly Xaa Xaa
Xaa Cys Xaa Xaa Gly Trp Xaa Gly Xaa Xaa Cys

(wherein Xaa may be any amino acid and Asx is either aspartic acid or asparagine).

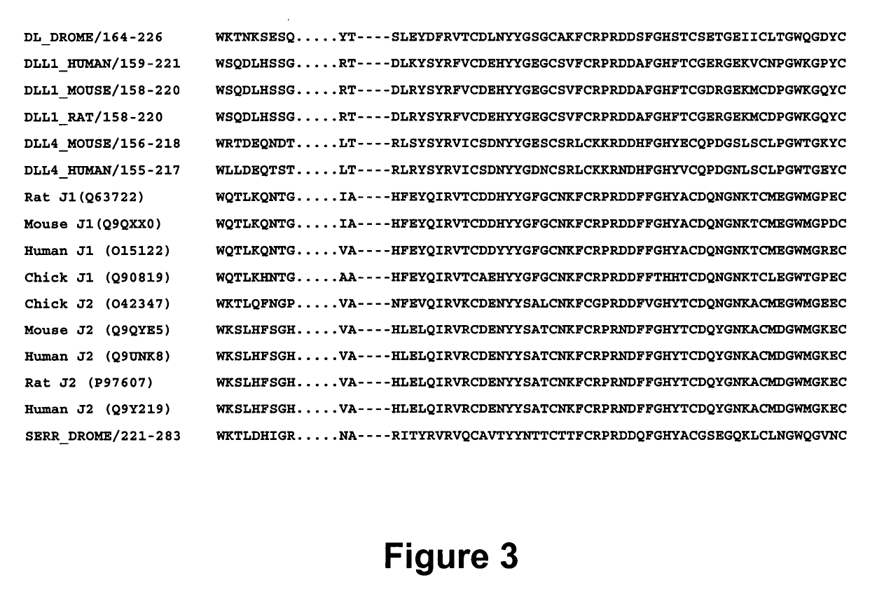

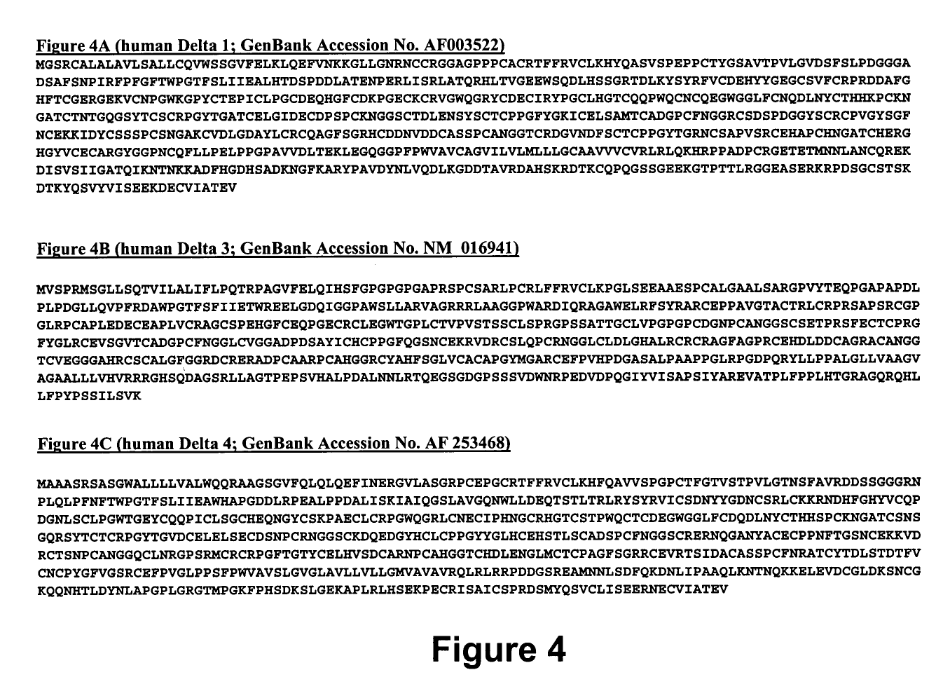

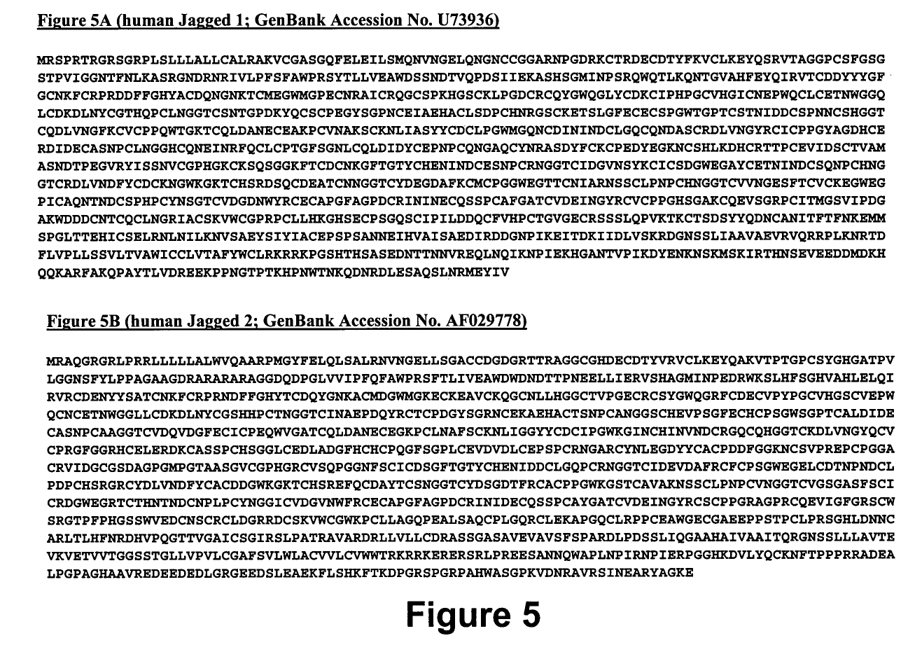

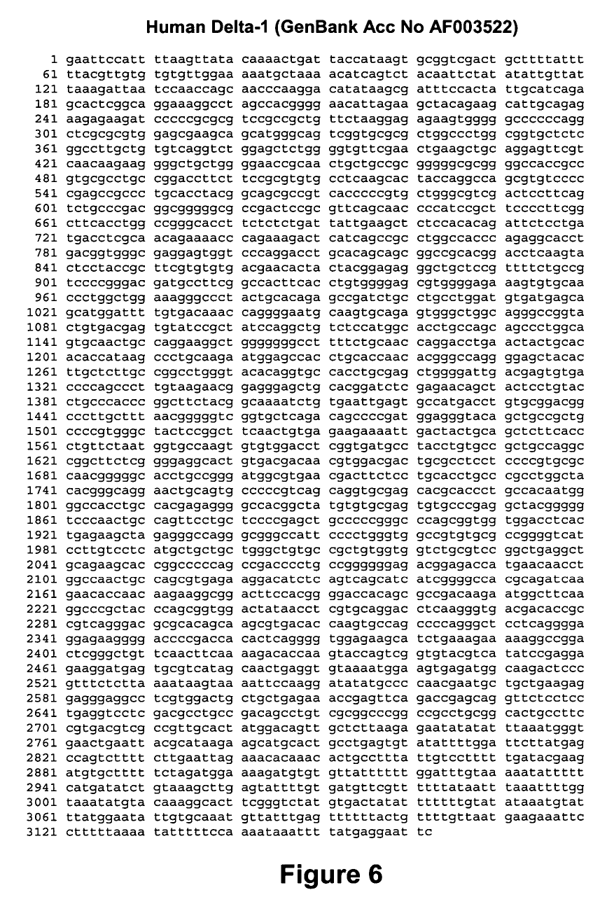

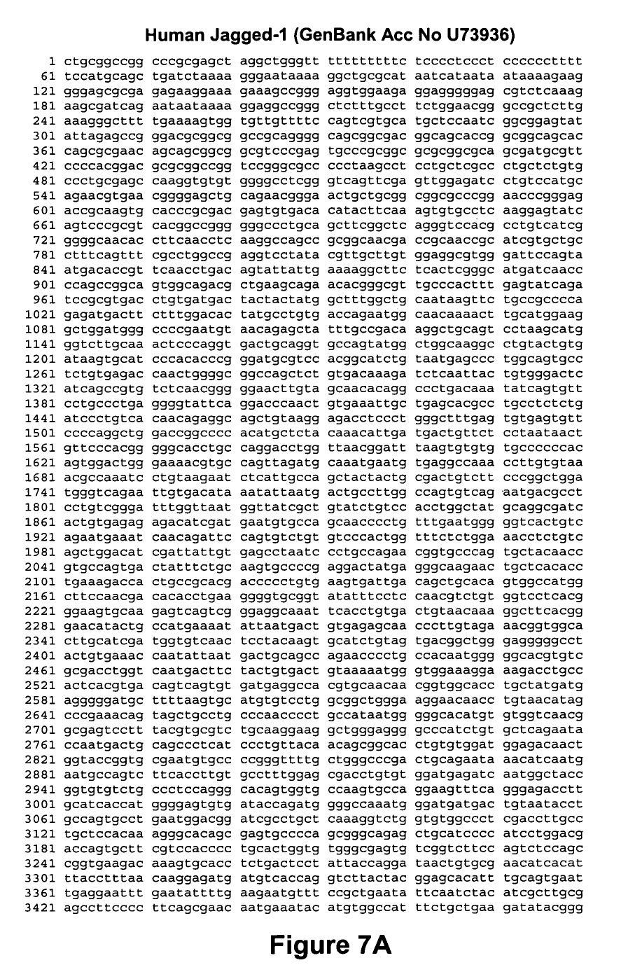

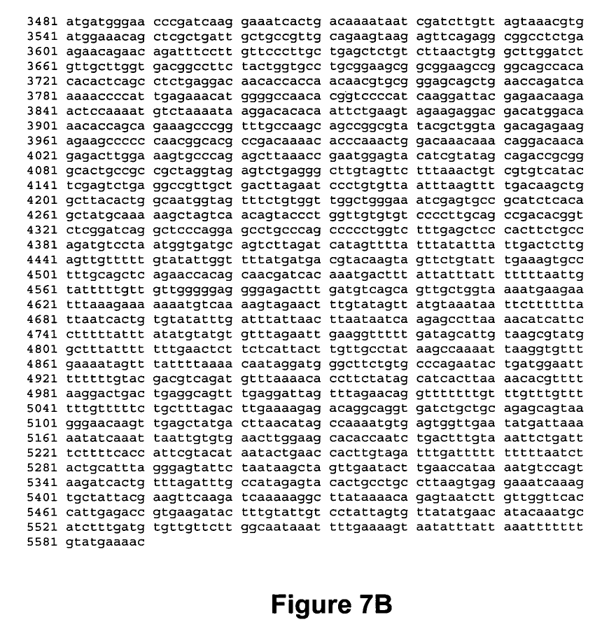

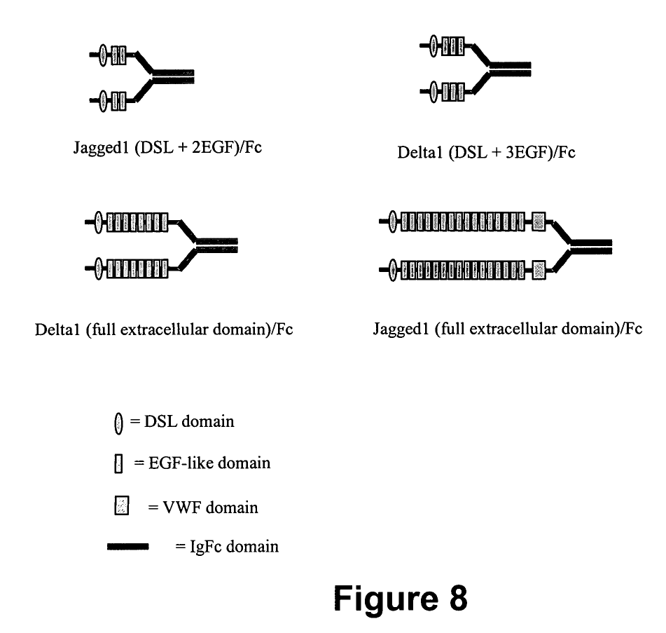

An alignment of DSL domains from Notch ligands from various sources is shown in FIG. 3.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors 1× and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain (SEQ ID NO:28): 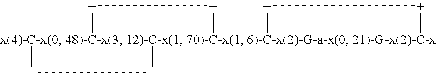
wherein:
‘ C’: conserved cysteine involved in a disulfide bond.
‘G’: often conserved glycine
‘a’: often conserved aromatic amino acid
‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

As a practical matter, whether any particular amino acid sequence is at least X% identical to another sequence can be determined conventionally using known computer programs. For example, the best overall match between a query sequence and a subject sequence, also referred to as a global sequence alignment, can be determined using a program such as, for example, the FASTDB computer program based on the algorithm of Brutlag et al. (Comp. App. Biosci. (1990) 6:237-245), or other algorithms described herein. In a sequence alignment the query and subject sequences are either both nucleotide sequences or both amino acid sequences. The result of the global sequence alignment may be given as percent identity.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

Suitably, for example, a Notch ligand N-terminal domain for use in the present invention may have at least 30%, preferably at least 50%, preferably at least 60%, preferably at least 70%, preferably at least 80%, preferably at least 90%, preferably at least 95% amino acid sequence identity to a Notch ligand N-terminal domain of human Jagged 1.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Jagged 2.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 1.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 3.

Alternatively a Notch ligand N-terminal domain for use in the present invention may, for example, have at least 30%, preferably at least 50%, preferably at least 60%, preferably at least 70%, preferably at least 80%, preferably at least 90%, preferably at least 95% amino acid sequence identity to a Notch ligand N-terminal domain of human Delta 4.

The term “heterologous amino acid sequence” or “heterologous nucleotide sequence” as used herein means a sequence which is not found in the native sequence (e.g. in the case of a Notch ligand sequence is not found in the native Notch ligand sequence) or its coding sequence. Typically, for example, such a sequence may be an IgFc domain or a tag such as a V5His tag.

Assays

Whether a substance can be used for modulating Notch signalling or activating a Notch receptor may be determined using any suitable screening assay, for example, as described in our co-pending International Patent Application published as WO 03/012441, as described in the Examples herein, or in Vamum-Finney et al, Journal of Cell Science 113, 4313-4318 (2000).

In addition, for example, Notch signalling can be monitored either through protein assays or through nucleic acid assays. Activation of the Notch receptor leads to the proteolytic cleavage of its cytoplasmic domain and the translocation thereof into the cell nucleus. The “detectable signal” referred to herein may be any detectable manifestation attributable to the presence of the cleaved intracellular domain of Notch. Thus, increased Notch signalling can be assessed at the protein level by measuring intracellular concentrations of the cleaved Notch domain. Activation of the Notch receptor also catalyses a series of downstream reactions leading to changes in the levels of expression of certain well defined genes. Thus, increased Notch signalling can be assessed at the nucleic acid level by say measuring intracellular concentrations of specific mRNAs. In one preferred embodiment of the present invention, the assay is a protein assay. In another preferred embodiment of the present invention, the assay is a nucleic acid assay.

The advantage of using a nucleic acid assay is that they are sensitive and that small samples can be analysed.

The intracellular concentration of a particular mRNA, measured at any given time, reflects the level of expression of the corresponding gene at that time. Thus, levels of mRNA of downstream target genes of the Notch signalling pathway can be measured in an indirect assay of the T-cells of the immune system. In particular, an increase in levels of Deltex, Hes-1 and/or IL-10 mRNA may, for instance, indicate induced anergy while an increase in levels of Dll-1 or IFN-γ mRNA, or in the levels of mRNA encoding cytokines such as IL-2, IL-5 and IL-13, may indicate improved responsiveness.

Various nucleic acid assays are known. Any convention technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

In particular, gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe. Those skilled in the art will readily envisage how these methods may be modified, if desired.

PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefore easily identifiable.

In general, reporter constructs useful for detecting Notch signalling by expression of a reporter gene may be constructed according to the general teaching of Sambrook et al (1989). Typically, constructs according to the invention comprise a promoter by the gene of interest, and a coding sequence encoding the desired reporter constructs, for example of GFP or luciferase. Vectors encoding GFP and luciferase are known in the art and available commercially.

Sorting of cells, based upon detection of expression of genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefore generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefore assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In vivo Expression Technology (IVET) (Camilli). IVET identifies genes up-regulated during say treatment or disease when compared to laboratory culture.

The advantage of using a protein assay is that Notch activation can be directly measured. Assay techniques that can be used to determine levels of a polypeptide are well known to those skilled in the art. Such assay methods include radioimmunoassays, competitive-binding assays, Western Blot analysis, antibody sandwich assays, antibody detection, FACS and ELISA assays.

As described above the modulator of Notch signalling may also be an immune cell which has been treated to modulate expression or interaction of Notch, a Notch ligand or the Notch signalling pathway. Such cells may readily be prepared, for example, as described in WO 00/36089 in the name of Lorantis Ltd, the text of which is herein incorporated by reference.

Cells of the Immune System

Antigen Presenting Cells

Where required, antigen-presenting cells (APCs) may be “professional” antigen presenting cells or may be another cell that may be induced to present antigen to T cells. Alternatively a APC precursor may be used which differentiates or is activated under the conditions of culture to produce an APC. An APC for use in the ex vivo methods of the invention is typically isolated from a tumour or peripheral blood found within the body of a patient. Preferably the APC or precursor is of human origin. However, where APCs are used in preliminary in vitro screening procedures to identify and test suitable nucleic acid sequences, APCs from any suitable source, such as a healthy patient, may be used.

APCs include dendritic cells (DCs) such as interdigitating DCs or follicular DCs, Langerhans cells, PBMCs, macrophages, B-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation and/or in combination of selected cytokine genes which would promote to immune potentiation (for example IL-2, IL-12, IFN-γ, TNF-α, IL-18 etc.). Such proteins include MHC molecules (Class I or Class II), CD80, CD86, or CD40. Most preferably DCs or DC-precursors are included as a source of APCs.

Dendritic cells (DCs) can be isolated/prepared by a number of means, for example they can either be purified directly from peripheral blood, or generated from CD34+precursor cells for example after mobilisation into peripheral blood by treatment with GM-CSF, or directly from bone marrow. From peripheral blood, adherent precursors can be treated with a GM-CSF/IL-4 mixture (Inaba K, et al. (1992) J. Exp. Med. 175: 1157-1167 (Inaba)), or from bone marrow, non-adherent CD34+cells can be treated with GM-CSF and TNF-a (Caux C, et al. (1992) Nature 360: 258-261 (Caux)). DCs can also be routinely prepared from the peripheral blood of human volunteers, similarly to the method of Sallusto and Lanzavecchia (Sallusto F and Lanzavecchia A (1994) J. Exp. Med. 179: 1109-1118) using purified peripheral blood mononucleocytes (PBMCs) and treating 2 hour adherent cells with GM-CSF and IL-4. If required, these may be depleted of CD19+B cells and CD3+, CD2+ T cells using magnetic beads (Coffin R S, et al. (1998) Gene Therapy 5: 718-722 (Coffin)). Culture conditions may include other cytokines such as GM-CSF or IL-4 for the maintenance and/or activity of the dendritic cells or other antigen presenting cells.

Thus, it will be understood that the term “antigen presenting cell or the like” as used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

T cells

Where required, T cells from any suitable source, such as a healthy patient, may be used and may be obtained from blood or another source (such as lymph nodes, spleen, or bone marrow). They may optionally be enriched or purified by standard procedures. The T cells may be used in combination with other immune cells, obtained from the same or a different individual. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used. It may also be convenient to use cell lines such as T cell hybridomas.

Introduction of Nucleic Acid Sequences into APCs and T-Cells

For ex-vivo applications, T-cells and APCs as described above are cultured in a suitable culture medium such as DMEM or other defined media, optionally in the presence of fetal calf serum.

Polypeptide substances may be administered to T-cells and/or APCs either in vivo or ex-vivo by introducing nucleic acid constructs/viral vectors encoding the polypeptide into cells under conditions that allow for expression of the polypeptide in the T-cell and/or APC. Similarly, nucleic acid constructs encoding antisense constructs may be introduced into the T-cells and/or APCs by transfection, viral infection or viral transduction.

In a preferred embodiment, nucleotide sequences encoding the enhancers of Notch ligand expression and/or activity will be operably linked to control sequences, including promoters/enhancers and other expression regulation signals.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) i.e. promoter.

It may also be advantageous for the promoters to be inducible so that the levels of expression of the heterologous gene can be regulated during the life-time of the cell. Inducible means that the levels of expression obtained using the promoter can be regulated.

Any of the above promoters may be modified by the addition of further regulatory sequences, for example enhancer sequences. Chimeric promoters may also be used comprising sequence elements from two or more different promoters.

Alternatively (or in addition), the regulatory sequences may be cell specific such that the gene of interest is only expressed in cells of use in the present invention. Such cells include, for example, APCs and T-cells.

The resulting T-cells and/or APCs that comprise nucleic acid constructs capable of up-regulating Notch ligand expression are now ready for use. If required, a small aliquot of cells may be tested for up-regulation of Notch ligand expression as described above. The cells may be prepared for administration to a patient or incubated with T-cells in vitro (ex vivo).

Assays of Immune Response and Tolerisation

Any of the assays described above (see “Assays”) can be adapted to monitor or to detect reduced reactivity, modified immune responses and/or tolerisation in immune cells, and to detect suppression and enhancement of immune responses for use in clinical applications.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity.

Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32 kD Disulfide linked Early Activation Antigen (EA-1) by 12-O-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of a serum such as fetal calf serum. Optimum cytokine concentrations may be determined by titration. One or more modulators of Notch signalling and interferons are then typically added to the culture medium together with the antigen (or antigenic determinant) of interest. The antigen may be added before, after or at substantially the same time as the substance(s). Cells are typically incubated with the substance(s) and antigen for at least one hour, preferably at least 3 hours, preferably at least 12 or at least 24 hours at approx 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142.

As discussed above, polypeptide substances may be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction. The resulting APCs that show increased levels of Notch signalling are now ready for use.

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s) to that used with the APC, then the T cell will not be brought into contact with the different substance(s) used in the APC. Alternatively, the T cell may be incubated with a first substance (or set of substances) to modulate Notch signalling, washed, resuspended and then incubated with the primed APC in the absence of both the substance(s) used to modulate the APC and the substance(s) used to modulate the T cell. Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Incubations will typically be for at least 1 hour, preferably at least 3 or 6 or 12 or 24 or more hours, in suitable culture medium at 37° C. Modification of immune responses, such as induction of immunotolerance may be determined by subsequently challenging T cells with antigen and measuring IL-2 production compared with control cells not exposed to APCs.

T cells or B cells which have been primed in this way may be used according to the invention to induce immunotolerance in other T cells or B cells.

Treatable Conditions

Preferably the modulation of the immune system is by control of T-cell activity. In particular, the present invention may be used for the treatment of T-cell mediated disease and infection. Diseased or infectious states that may be described as being mediated by T cells include, but are not limited to, any one or more of asthma, allergy, graft rejection, autoimmunity, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple schlerosis, rheumatoid arthritis and diabetes. The present invention may also be used in organ transplantation or bone marrow transplantation.

As indicated above, the present invention is useful in treating immune disorders such as autoimmune diseases or graft rejection such as allograft rejection.

For example, the present invention may be used for the treatment of organ transplants (e.g. kidney, heart, lung, liver or pancreas transplants), tissue transplants (e.g. skin grafts) or cell transplants (e.g. bone marrow transplants or blood transfusions).

A brief overview of the most common types of organ and tissue transplants is set out below.

1. Kidney Transplants:

Kidneys are the most commonly transplanted organs. Kidneys can be donated by both cadavers and living donors and kidney transplants can be used to treat numerous clinical indications (including diabetes, various types of nephritis and kidney failure). Surgical procedure for kidney transplantation is relatively simple. However, matching blood types and histocompatibility groups is desirable to avoid graft rejection. It is indeed important that a graft is accepted as many patients can become “sensitised” after rejecting a first transplant. Sensitisation results in the formation of antibodies and the activation of cellular mechanisms directed against kidney antigens. Thus, any subsequent graft containing antigens in common with the first is likely to be rejected. As a result, many kidney transplant patients must remain on some form of immunosuppressive treatment for the rest of their lives, giving rise to complications such as infection and metabolic bone disease.

2. Heart Transplantation

Heart transplantation is a very complex and high-risk procedure. Donor hearts must be maintained in such a manner that they will begin beating when they are placed in the recipient and can therefore only be kept viable for a limited period under very specific conditions. They can also only be taken from brain-dead donors. Heart transplants can be used to treat various types of heart disease and/or damage. HLA matching is obviously desirable but often impossible because of the limited supply of hearts and the urgency of the procedure.

3. Lung Transplantation

Lung transplantation is used (either by itself or in combination with heart transplantation) to treat diseases such as cystic fibrosis and acute damage to the lungs (e.g. caused by smoke inhalation). Lungs for use in transplants can only be recovered from brain-dead donors.

4. Pancreas Transplantation

Pancreas transplantation is mainly used to treat diabetes mellitus, a disease caused by malfunction of insulin-producing islet cells in the pancreas. Organs for transplantation can only be recovered from cadavers although it should be noted that transplantation of the complete pancreas is not necessary to restore the function needed to produce insulin in a controlled fashion. Indeed, transplantation of the islet cells alone could be sufficient. Because kidney failure is a frequent complication of advanced diabetes, kidney and pancreas transplants are often carried out simultaneously.

5. Skin Grafting

Most skin transplants are done with autologous tissue. However, in cases of severe burning (for example), grafts of foreign tissue may be required (although it should be noted that these grafts are generally used as biological dressings as the graft will not grow on the host and will have to be replaced at regular intervals). In cases of true allogenic skin grafting, rejection may be prevented by the use of immunosuppressive therapy. However, this leads to an increased risk of infection and is therefore a major drawback in burn victims.

6. Liver Transplantation

Liver transplants are used to treat organ damage caused by viral diseases such as hepititis, or by exposure to harmful chemicals (e.g. by chronic alcoholism). Liver transplants are also used to treat congenital abnormalities. The liver is a large and complicated organ meaning that transplantation initially posed a technical problem. However, most transplants (65%) now survive for more than a year and it has been found that a liver from a single donor may be split and given to two recipients. Although there is a relatively low rate of graft rejection by lung transplant patients, leukocytes within the donor organ together with anti-blood group antibodies can mediate antibody-dependent hemolysis of recipient red blood cells if there is a mismatch of blood groups. In addition, manifestations of GVHD have occurred in liver transplants even when donor and recipient are blood-group compatible.

Examples of disorders that may be treated include a group commonly called autoimmune diseases. The spectrum of autoimmune disorders ranges from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail: Organ-specific autoimmune diseases include multiple sclerosis, insulin dependent diabetes mellitus, several forms of anemia (aplastic, hemolytic), autoimmune hepatitis, thyroiditis, insulitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is also useful in cancer therapy. The present invention is especially useful in relation to adenocarcinomas such as: small cell lung cancer, and cancer of the kidney, uterus, prostrate, bladder, ovary, colon and breast.

Pharmaceutical Compositions

Preferably the active agents (modulators of Notch signalling and interferons, polynucleotides coding for interferons and/or interferon inducers) of the present invention are administered in the form of pharmaceutical compositions. The pharmaceutical compositions may be for human or animal usage in human and veterinary medicine and in addition to one or more active agents will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s). Preservatives, stabilizers, dyes and even flavoring agents may also be provided in such a pharmaceutical composition. Examples of preservatives include sodium benzoate, sorbic acid and esters of p-hydroxybenzoic acid. Antioxidants and suspending agents may be also used.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

In one embodiment the therapeutic agents used in the present invention may be administered directly to patients in vivo. Alternatively or in addition, the agents may be administered to cells (such as T cells and/or APCs or stem or tissue cells) in an ex vivo manner. For example, leukocytes such as T cells or APCs may be obtained from a patient or donor in known manner, treated/incubated ex vivo in the manner of the present invention, and then administered to a patient.

In general, a therapeutically effective daily dose may for example range from 0.01 to 500 mg/kg, for example 0.01 to 50 mg/kg body weight of the subject to be treated, for example 0.1 to 20 mg/kg. The agents of the present invention may also be administered by intravenous infusion, at a dose which is likely to range from for example 0.001-10 mg/kg/hr.

A skilled practitioner will be able to determine readily the optimum route of administration and dosage for any particular patient depending on, for example, the age, weight and condition of the patient. Preferably the pharmaceutical compositions are in unit dosage form.

The agents of the present invention can be administered by any suitable means including, but not limited to, for example, oral, rectal, nasal, topical (including intradermal, transdermal, aerosol, buccal and sublingual), vaginal and parenteral (including subcutaneous, intramuscular, intravenous and intradermal) routes of administration.

Suitably the active agents are administered in combination with a pharmaceutically acceptable carrier or diluent as described under the heading “Pharmaceutical compositions” above. The pharmaceutically acceptable carrier or diluent may be, for example, sterile isotonic saline solutions, or other isotonic solutions such as phosphate-buffered saline. The agents of the present invention may suitably be admixed with any suitable binder(s), lubricant(s), suspending agent(s), coating agent(s), solubilising agent(s).

In one embodiment, it may be desired to formulate the compound in an orally active form. Thus, for some applications, active agents may be administered orally in the form of tablets containing excipients such as starch or lactose, or in capsules or ovules either alone or in admixture with excipients, or in the form of elixirs, solutions or suspensions containing flavouring or colouring agents. Doses such as tablets or capsules comprising the agents may be administered singly or two or more at a time, as appropriate. It is also possible to administer the active agents in sustained release formulations.

Alternatively or in addition, active agents may be administered by inhalation, intranasally or in the form of aerosol, or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin, for example at a concentration of between 1 and 10% by weight, into an ointment consisting of a white wax or white soft paraffin base together with such stabilisers and preservatives as may be required.

Active agents such as polynucleotides and proteins/polypeptides may also be administered by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include, but are not limited to, mucosal, nasal, oral, parenteral, gastrointestinal, topical, or sublingual routes. Active agents may also be adminstered by needleless systems, such as ballistic delivery on particles for delivery to the epidermis or dermis or other sites such as mucosal surfaces.

Active agents may also be injected parenterally, for example intracavernosally, intravenously, intramuscularly or subcutaneously.

For parenteral administration, active agents may for example be used in the form of a sterile aqueous solution which may contain other substances, for example enough salts or monosaccharides to make the solution isotonic with blood.

For buccal or sublingual administration, agents may for example be administered in the form of tablets or lozenges which can be formulated in a conventional manner.

For oral, parenteral, buccal and sublingual administration to subjects (such as patients), the dosage level of active agents and their pharmaceutically acceptable salts and solvates may typically be from 10 to 500 mg (in single or divided doses). Thus, and by way of example, tablets or capsules may contain from 5 to 100 mg of active agent for administration singly, or two or more at a time, as appropriate. As indicated above, the physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

The routes of administration and dosages described are intended only as a guide since a skilled practitioner will be able to determine readily the optimum route of administration and dosage for any particular patient depending on, for example, the age, weight and condition of the patient.

The term treatment or therapy as used herein should be taken to encompass diagnostic and prophylatic applications.

The treatment of the present invention includes both human and veterinary applications.

The active agents of the present invention may also be administered with other active agents such as, for example, immunosuppressants, steroids or anticancer agents.

Where treated ex-vivo, modified cells of the present invention are preferably administered to a host by direct injection into the lymph nodes of the patient. Typically from 104 to 108 treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Imnmunol. Meth., 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibodycoated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

Combination Treatments

Combination treatments wherein active agents of the present invention are administered in combination with other active agents, antigens or antigenic determinants are also within the scope of the present invention.

By “simultaneously” is meant that the active agents are administered at substantially the same time, and preferably together in the same formulation.

By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours, and preferably within less than about one to about four hours. When administered contemporaneously, the agents are preferably administered at the same site on the animal. The term “same site” includes the exact location, but can be within about 0.5 to about 15 centimeters, preferably from within about 0.5 to about 5 centimeters.

The term “separately” as used herein means that the agents are administered at an interval, for example at an interval of about a day to several weeks or months. The active agents may be administered in either order.

The term “sequentially” as used herein means that the agents are administered in sequence, for example at an interval or intervals of minutes, hours, days or weeks. If appropriate the active agents may be administered in a regular repeating cycle.

It will be appreciated that in one embodiment the therapeutic agents used in the present invention may be administered directly to patients in vivo. Alternatively or in addition, the agents may be administered to immune cells such as T cells and/or APCs in an ex vivo manner. For example, leukocytes such as T cells or APCs may be obtained from a patient or donor in known manner, treated/incubated ex vivo in the manner of the present invention, and then administered to a patient. In addition, it will be appreciated that a combination of routes of administration may be employed if desired. For example, where appropriate one component (such as the modulator of Notch signalling) may be administered ex-vivo and the other may be administered in vivo, or vice versa.

Preferably the active agents are administered topically, intranasally or parenterally (e.g. sub-cutaneously, intradermally or intramuscularly), preferably to the same area, and one or more may be administered in a sustained release form.

Chemical Cross-Linking

Chemically coupled (cross-linked) sequences can be prepared from individual protein sequences and coupled using known chemical coupling techniques. A conjugate can for example be assembled using conventional solution- or solid-phase peptide synthesis methods, affording a fully protected precursor with only the terminal amino group in deprotected reactive form. This function can then be reacted directly with, for example, a protein for Notch signalling modulation or a suitable reactive derivative thereof. Alternatively, this amino group may be converted into a different functional group suitable for reaction with a cargo moiety or a linker. Thus, e.g. reaction of the amino group with succinic anhydride will provide a selectively addressable carboxyl group, while further peptide chain extension with a cysteine derivative will result in a selectively addressable thiol group. Once a suitable selectively addressable functional group has been obtained in the delivery vector precursor, a protein for Notch signalling modulation or a derivative thereof may be attached through e.g. amide, ester, or disulphide bond formation. Cross-linking reagents which can be utilized are discussed, for example, in Means, G. E. and Feeney, R. E., Chemical Modification of Proteins, Holden-Day, 1974, pp. 39-43.

Modulators of Notch signalling modulation may if desired be linked directly or indirectly suitably via a linker moiety. Direct linkage may occur through any convenient functional group on the modulator (e.g. protein for Notch signalling modulation) such as a thiol, hydroxy, carboxy or amino group. Indirect linkage which is may sometimes be preferable, will occur through a linking moiety. Suitable linking moieties include bi- and multi-functional alkyl, aryl, aralkyl or peptidic moieties, alkyl, aryl or aralkyl aldehydes acids esters and anyhdrides, sulphydryl or carboxyl groups, such as maleimido benzoic acid derivatives, maleimido proprionic acid derivatives and succinimido derivatives or may be derived from cyanuric bromide or chloride, carbonyldiimidazole, succinimidyl esters or sulphonic halides and the like.

Modified/Humanised Antibodies

Preferably, antibodies for use to treat human patients will be chimeric or humanised antibodies. Antibody “humanisation” techniques are well known in the art. These techniques typically involve the use of recombinant DNA technology to manipulate DNA sequences encoding the polypeptide chains of the antibody molecule.

As described in U.S. Pat. No. 5,859,205 early methods for humanising monoclonal antibodies (Mabs) involved production of chimeric antibodies in which an antigen binding site comprising the complete variable domains of one antibody is linked to constant domains derived from another antibody. Such chimerisation procedures are described in EP-A-0120694 (Celltech Limited), EP-A-0125023 (Genentech Inc. and City of Hope), EP-A-0 171496 (Res. Dev. Corp. Japan), EP-A-0 173 494 (Stanford University), and WO 86/01533 (Celltech Limited). For example, WO 86/01533 discloses a process for preparing an antibody molecule having the variable domains from a mouse MAb and the constant domains from a human immunoglobulin.

In an alternative approach, described in EP-A-0239400 (Winter), the complementarity determining regions (CDRs) of a mouse MAb are grafted onto the framework regions of the variable domains of a human immunoglobulin by site directed mutagenesis using long oligonucleotides. Such CDR-grafted humanised antibodies are much less likely to give rise to an anti-antibody response than humanised chimeric antibodies in view of the much lower proportion of non-human amino acid sequence which they contain. Examples in which a mouse MAb recognising lysozyme and a rat MAb recognising an antigen on human T-cells were humanised by CDR-grafting have been described by Verhoeyen et al (Science, 239, 1534-1536, 1988) and Riechmann et al (Nature, 332, 323-324, 1988) respectively. The preparation of CDR-grafted antibody to the antigen on human T cells is also described in WO 89/07452 (Medical Research Council).

In WO 90/07861 Queen et al propose four criteria for designing humanised immunoglobulins. The first criterion is to use as the human acceptor the framework from a particular human immunoglobulin that is unusually homologous to the non-human donor immunoglobulin to be humanised, or to use a consensus framework from many human antibodies. The second criterion is to use the donor amino acid rather than the acceptor if the human acceptor residue is unusual and the donor residue is typical for human sequences at a specific residue of the framework. The third criterion is to use the donor framework amino acid residue rather than the acceptor at positions immediately adjacent to the CDRs. The fourth criterion is to use the donor amino acid residue at framework positions at which the amino acid is predicted to have a side chain atom within about 3 A of the CDRs in a three-dimensional immunoglobulin model and to be capable of interacting with the antigen or with the CDRs of the humanised immunoglobulin. It is proposed that criteria two, three or four may be applied in addition or alternatively to criterion one, and may be applied singly or in any combination.

Antigens and Allergens

In one embodiment, the agents of the present invention may be administered in simultaneous, separate or sequential combination with antigens or antigenic determinants (or polynucleotides coding therefor), to modify (increase or decrease) the immune response to such antigens or antigenic determinants.

An antigen suitable for use in the present invention may be any substance that can be recognised by the immune system, and is generally recognised by an antigen receptor. Preferably the antigen used in the present invention is an immunogen. An allergic response occurs when the host is re-exposed to an antigen that it has encountered previously.

The immune response to antigen is generally either cell mediated (T cell mediated killing) or humoral (antibody production via recognition of whole antigen). The pattern of cytokine production by TH cells involved in an immune response can influence which of these response types predominates: cell mediated immunity (TH1) is characterised by high IL-2 and IFNγ but low IL-4 production, whereas in humoral immunity (TH2) the pattern is low IL-2 and IFNγ but high IL-4, IL-5 and IL-13. Since the secretory pattern is modulated at the level of the secondary lymphoid organ or cells, then pharmacological manipulation of the specific TH cytokine pattern can influence the type and extent of the immune response generated.

The TH1-TH2 balance refers to the relative representation of the two different forms of helper T cells. The two forms have large scale and opposing effects on the immune system. If an immune response favours TH1 cells, then these cells will drive a cellular response, whereas TH2 cells will drive an antibody-dominated response. The type of antibodies responsible for some allergic reactions is induced by TH2 cells.

The antigen or allergen (or antigenic determinant thereof) used in the present invention may be a peptide, polypeptide, carbohydrate, protein, glycoprotein, or more complex material containing multiple antigenic epitopes such as a protein complex, cell-membrane preparation, whole cells (viable or non-viable cells), bacterial cells or virus/viral component. In particular, it is preferred to use antigens known to be associated with autoimmune diseases such as myelin basic protein (associated with multiple sclerosis), collagen (associated with rheumatoid arthritis), and insulin (diabetes), or antigens associated with rejection of non-self tissue such as MHC antigens or antigenic determinants thereof. Where primed the APCs and/or T cells of the present invention are to be used in tissue transplantation procedures, antigens may be obtained from the tissue donor. Polynucleotides coding for antigens or antigenic determinants which may be expessed in a subject may also be used.

### EXAMPLES

Various preferred features and embodiments of the present invention will now be described in more detail by way of non-limiting examples.

### Example 1

### hDelta1-IgG4Fc Fusion Protein

A fusion protein comprising the extracellular domain of human Delta1 fused to the Fc domain of human IgG4 (“hDelta1-IgG4Fc”) was prepared by inserting a nucleotide sequence coding for the extracellular domain of human Delta1 (see, e.g. Genbank Accession No AF003522) into the expression vector pCONγ (Lonza Biologics, Slough, UK) and expressing the resulting construct in CHO cells. The amino acid sequence of the resulting expressed fusion protein was as follows (SEQ ID NO:28):

MGSRCALALAVLSALLCQVWSSGVFELKLQEFVNKKGLLGNRNCCRGGAG
PPPCACRTFFRVCLKHYQASVSPEPPCTYGSAVTPVLGVDSFSLPDGGGA
DSAFSNPIRFPFGFTWPGTFSLIIEALHTDSPDDLATENPERLISRLATQ
RHLTVGEEWSQDLHSSGRTDLKYSYRFVCDEHYYGEGCSVFCRPRDDAFG
HFTCGERGEKVCNPGWKGPYCTEPICLPGCDEQHGFCDKPGECKCRVGWQ
GRYCDECIRYPGCLHGTCQQPWQCNCQEGWGGLFCNQDLNYCTHHKPCKN
GATCTNTGQGSYTCSCRPGYTGATCELGIDECDPSPCKNGGSCTDLENSY
SCTCPPGFYGKICELSAMTCADGPCFNGGRCSDSPDGGYSCRCPVGYSGF
NCEKKIDYCSSSPCSNGAKCVDLGDAYLCRCQAGFSGRLHCDDNVDDCAS
SPCANGGTCRDGVNDFSCTCPPGYTGRNCSAPVSRCEHAPCHNGATCHER
GHGYVCECARGYGGPNCQFLLPELPPGPAVVDLTEKLEASTKGPSVFPLA
PCSRSTSESTAALGCLVKDYFPEPVTVSWNSGALTSGVHTFPAVLQSSGL
YSLSSVVTVPSSSLGTKTYTCNVDHKPSNTKVDKRVESKYGPPCPSCPAP
EFLGGPSVFLFPPKPKDTLMISRTPEVTCVVVDVSQEDPEVQFNWYVDGV
EVHNAKTKPREEQFNSTYRVVSVLTVLHQDWLNGKEYKCKVSNKGLPSSI
EKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEAL
HNHYTQKSLSLSLGK

Wherein the first underlined sequence is the signal peptide (cleaved from the mature protein) and the second, underlined sequence is the IgG4 Fc sequence. The protein normally exists as a dimer linked by cysteine disulphide bonds (see e.g. schematic representation in Figures hereto).

### Example 2

### Dynabeads ELISA Assay Method For Detecting Notch Signalling Activity in the Presence of Rapamycin

(i) CD4+ Cell Purification

Spleens were removed from mice (variously Balb/c females, 8-10 weeks, C57B/6 females, 8-10 weeks, D011.10 transgenic females, 8-10 weeks) and passed through a 0.2 μM cell strainer into 20 ml R10F medium (R10F—RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

96 well flat-bottomed plates were coated with DPBS plus 1 μg/ml anti-hamsterIgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus hDelta1-IgG4Fc fusion protein (10 μg/ml; as described above). The plates were incubated for 2-3 hours at 37° C. then washed again with DPBS before cells (prepared as described above) were added.

(iii) Primaryy Polyclonal Stimulation and ELISA

CD4+ cells were cultured in 96 well, flat-bottomed plates pre-coated according to (ii) above. Cells were re-suspended, following counting, at 2×10⁶/ml in R10F medium plus 4 μg/ml anti-CD28 antibody (Pharmingen, Cat No 553294, Clone No 37.51). 1001 μl cell suspension was added per well. 100 μl of R10F medium was then added to each well to give a final volume of 200 μl (2×10⁵ cells/well, anti-CD28 final concentration 2 μg/ml) Rapamycin (Sigma) was added from a stock solution (4 mM in DMSO) to give final concentrations from 0 to 5000 nM. The plates were then incubated at 37° C. for 72 hours.

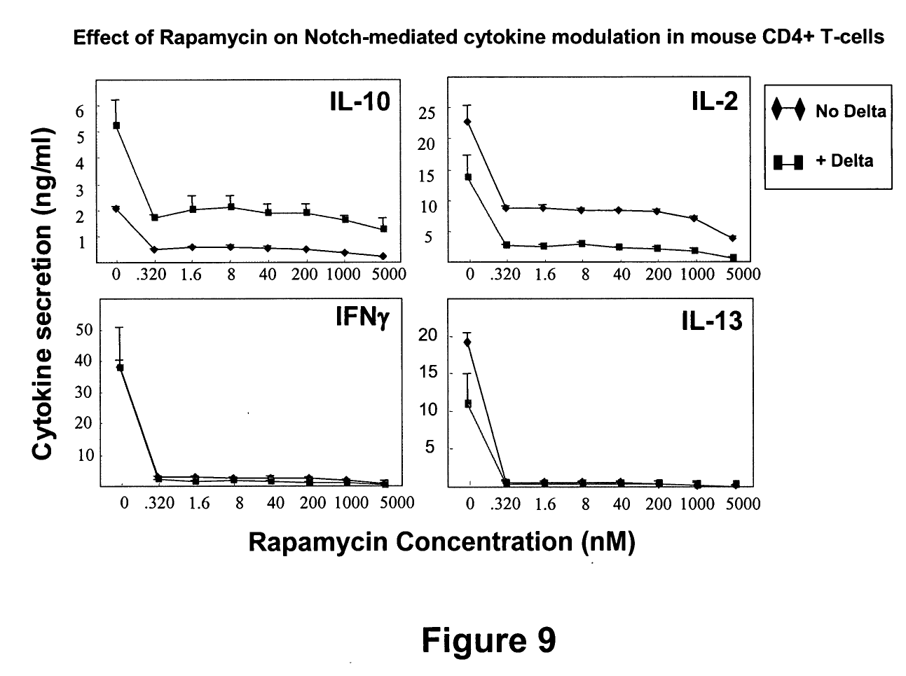

125 μl supernatant was then removed from each well and stored at −20° C. until tested by ELISA for IL-2, IL-10, IFNg and IL-13 using antibody pairs from R & D Systems (Abingdon, UK). Results are shown in FIG. 9.

### Example 3

### Dynabeads ELISA Assay Method For Detecting Notch Signalling Activity in the Presence of Cyclosporin A

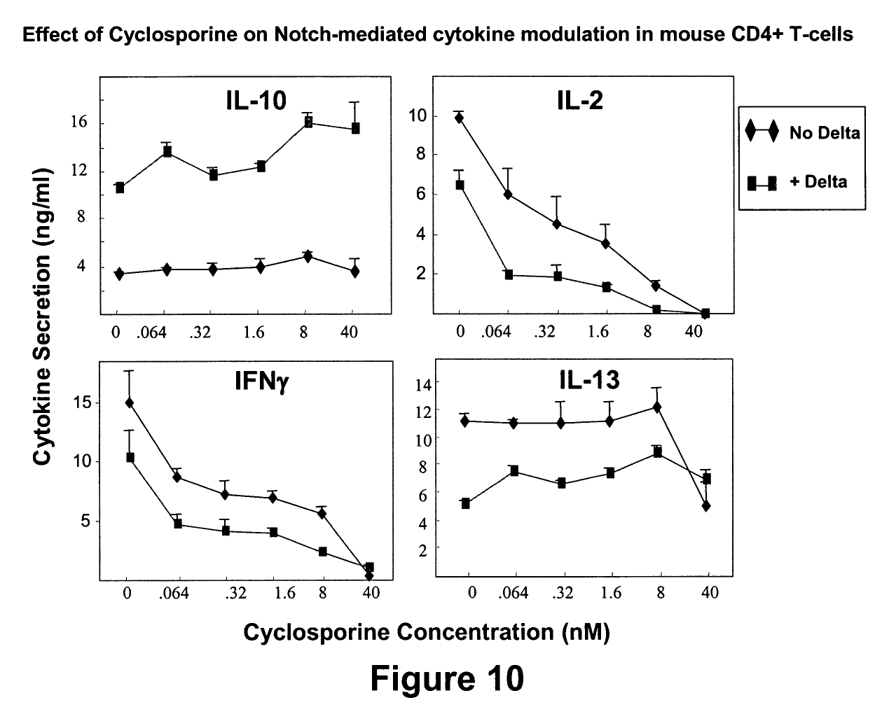

The procedure of Example 2 was repeated except that cyclosporin A (Sigma) was added (from a stock solution of 4 mM in ethanol) in place of rapamycin to give final concentrations of from 0 to 40 nM. Results are shown in FIG. 10.

### Example 4

### Dynabeads ELISA Assay Method For Detecting Notch Signalling Activity in the Presence of FK-506

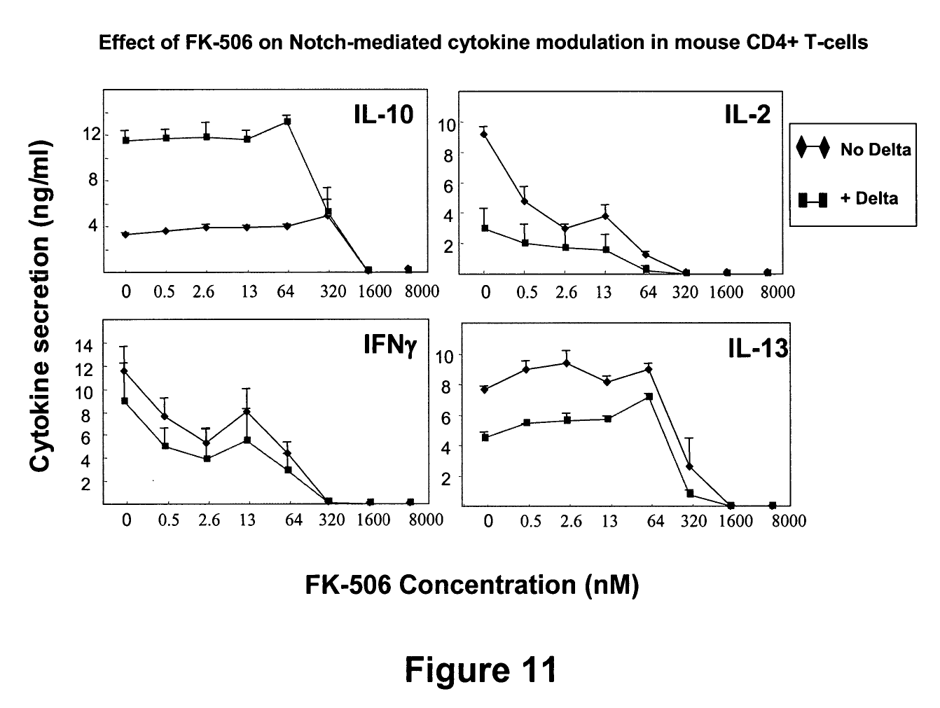

The procedure of Example 2 was repeated except that FK-506 (Calbiochem) was added (from a stock solution of 4 mM in DMSO) in place of rapamycin to give final concentrations of from 0 to 8000 nM. Results are shown in FIG. 11.

### Example 5

### Dynabeads ELISA Assay Method For Detecting Notch Signalling Activity in the Presence of Dexamethasone

(i) CD4+ Cell Purification

Spleens were removed from mice (variously Balb/c females, 8-10 weeks, C57B/6 females, 8-10 weeks, D011.10 transgenic females, 8-10 weeks) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0 M KHCO₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

96 well flat-bottomed plates were coated with DPBS plus 1 μg/ml anti-hamsterIgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus hDelta1-IgG4Fc fusion protein (10 μg/ml; as described above). The plates were incubated for 2-3 hours at 37° C. then washed again with DPBS before cells (prepared as described above) were added.

(iii) Primary Polyclonal Stimulation and ELISA

CD4+ cells were cultured in 96 well, flat-bottomed plates pre-coated according to (ii) above. Cells were re-suspended, following counting, at 2×10⁶/ml in R10F medium plus 4 μg/ml anti-CD28 antibody (Pharmingen, Cat No 553294, Clone No 37.51). 100 μl cell suspension was added per well. 100 μl of R10F medium was then added to each well to give a final volume of 200 μl (2×10⁵ cells/well, anti-CD28 final concentration 2 μg/ml) Dexamethasone was added from a stock solution (4 mM in DMSO) to give final concentrations from 0 to 5000 nM. The plates were then incubated at 37° C. for 72 hours.

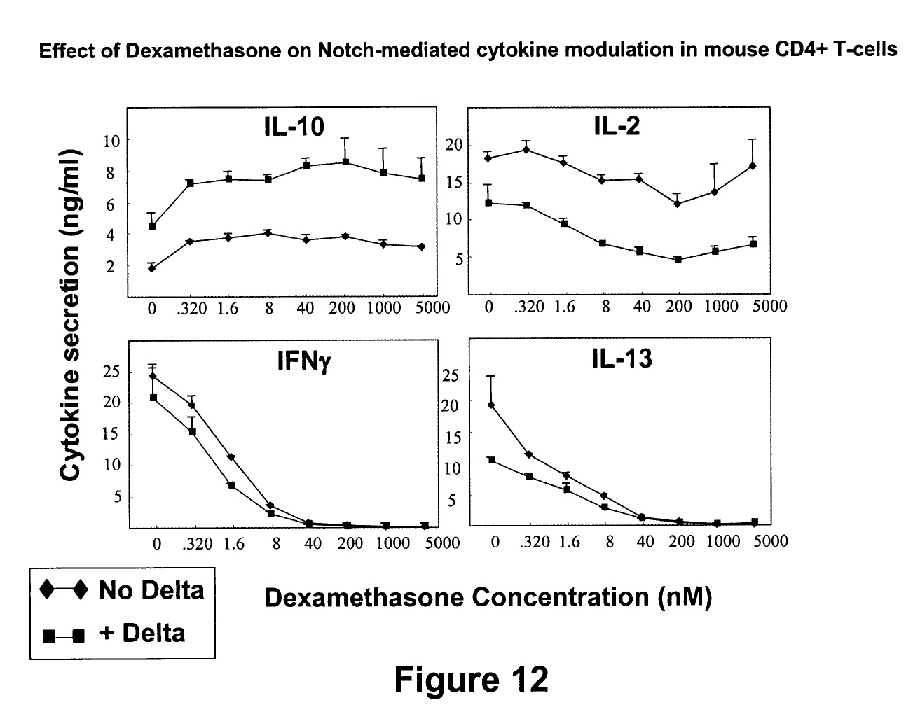

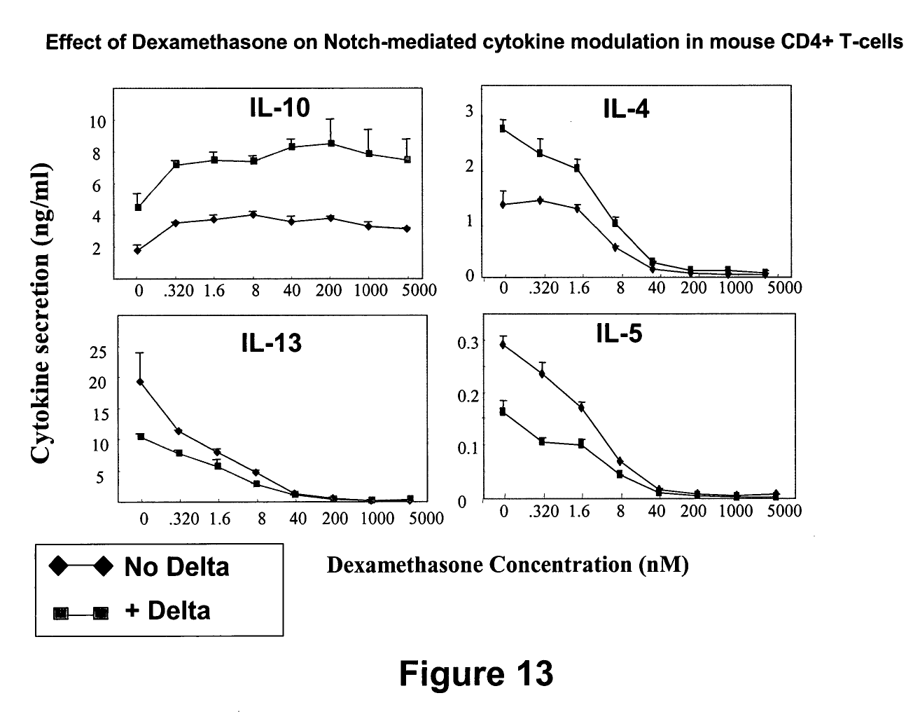

125 μl supernatant was then removed from each well and stored at −20° C. until tested by ELISA for IL-2, IL-10, IFNg and IL-13 using antibody pairs from R & D Systems (Abingdon, UK). Results are shown in FIG. 12. Further results with IL-4 and IL-5 are shown in FIG. 13.

### Example 6

### Luciferase Assay for Detecting Notch Signalling Activity

hDelta1-IgG4Fc fusion protein (Example 1) was immobilised on Streptavidin-Dynabeads (CELLection Biotin Binder Dynabeads [Cat. No. 115.21] at 4.0×10⁸ beads/ml from Dynal (UK) Ltd; “beads”) in combination with biotinylated α-IgG-4 (clone JDC14 at 0.5 mg/ml from Pharmingen [Cat. No. 555879]) as follows:

1×10⁷ beads (25 μl of beads at 4.0×10⁸ beads/ml) and 2 μg biotinylated α-IgG-4 was used for each sample assayed. PBS was added to the beads to 1 ml and the mixture was spun down at 13,000 rpm for 1 minute. Following washing with a further 1 ml of PBS the mixture was spun down again. The beads were then resuspended in a final volume of 100 PI of PBS containing the biotinylated α-IgG-4 in a sterile Eppendorf tube and placed on a shaker at room temperature for 30 minutes. PBS to was added to 1 ml and the mixture was spun down at 13,000 rpm for 1 minute and then washed twice more with 1 ml of PBS.

The mixture was then spun down at 13,000 rpm for 1 minute and the beads were resupsended in 50 μl PBS per sample. 50 μl of biotinylated α-IgG-4-coated beads were added to each sample and the mixture was incubated on a rotary shaker at 4° C. overnight. The tube was then spun at 1000 rpm for 5 minutes at room temperature.

The beads then were washed with 10 ml of PBS, spun down, resupended in 1 ml of PBS, transferred to a sterile Eppendorf tube, washed with a further 2×1 ml of PBS, spun down and resuspended in a final volume of 100 μl of DMEM plus 10% (HI)FCS plus glutamine plus P/S, i.e. at 1.0×10⁵ beads/μl.

N27#11 cells (CHO cells expressing full length human Notch2 and a CBF1-luciferase reporter construct; T₈₀ flask; as described in WO 03/012441, Lorantis, e.g. see Example 7 therein) were removed using 0.02% EDTA (with trypsin) solution (Sigma), spun down and resuspended in 10 ml DMEM plus 10% (HI)FCS plus glutamine plus P/S. 10 μl of cells were counted and the cell density was adjusted to 1.0×10⁵ cells/ml with fresh DMEM plus 10% (HI)FCS plus glutamine plus P/S. 1.0×10⁵ of the cells were plated out per well of a 24-well plate in a 1 ml volume of DMEM plus 10% (HI)FCS plus glutamine plus P/S and cells were placed in an incubator to settle down for at least 30 minutes.

20 μl of beads were then added in duplicate to a pair of wells to give 2.0×10⁶ beads/well (100 beads/cell). The plate was left in a CO₂ incubator overnight.

Supernatant was then removed from all the wells, 100 μl of SteadyGlo™ luciferase assay reagent (Promega) was added and the resulting mixture left at room temperature for 5 minutes.

The mixture was then pipetted up and down 2 times to ensure cell lysis and the contents from each well were transferred to a 96 well plate (with V-shaped wells) and spun in a plate holder for 5 minutes at 1000 rpm at room temperature.

175 μl of cleared supernatant was then transferred to a white 96-well plate (Nunc) leaving the beads pellet behind.

Luminescence was then read in a TopCount™ (Packard) counter.

The invention is further described by the following numbered paragraphs:

1. A product comprising:
i) a modulator of the Notch signalling pathway; and
ii) an immunosuppressive agent;
as a combined preparation for simultaneous, contemporaneous, separate or sequential use for modulation of the immune system.

2. A product as described in paragraph 1 for modulation of T cell activity.

3. A product as described in paragraph 1 or paragraph 2 for the treatment of inflammation, asthma, allergy, graft rejection, graft-versus-host disease or autoimmune disease.

4. A product as described in any one of the preceding paragraphs wherein the modulator of the Notch signalling pathway comprises Delta or Jagged or a fragment, derivative, homologue, analogue or allelic variant thereof or a polynucleotide coding for Delta, Jagged or a fragment, derivative, homologue, analogue or allelic variant thereof.

5. A product as described in any one of paragraphs 1 to 4 wherein the modulator of the Notch signalling pathway comprises a fusion protein comprising a segment of a Notch ligand extracellular domain and an immunoglobulin F_{c} segment or a polynucleotide coding for such a fusion protein.

6. A product as described in any one of paragraphs 1 to 4 wherein the modulator of the Notch signalling pathway comprises a protein or polypeptide comprising a DSL or EGF-like domain or a polynucleotide sequence coding for such a protein.

7. A product as described in paragraph 6 wherein the modulator of the Notch signalling pathway comprises a protein or polypeptide comprising a Notch ligand DSL domain at least one EGF-like domain or a polynucleotide sequence coding for such a protein or polypeptide.

8. A product as described in any one of paragraphs 1 to 3 wherein modulator of the Notch signalling pathway comprises Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

9. A product as described in any one of paragraphs 1 to 3 wherein the modulator of the Notch signalling pathway comprises a dominant negative version of a Notch signalling repressor, or a polynucleotide which codes for a dominant negative version of a Notch signalling repressor.

10. A product as described in any one of the preceding paragraphs wherein the immunosuppressive agent is an immunologically active steroid or steroid derivative.

11. A product as described in any one of paragraphs 1 to 9 wherein the immunosuppressive agent is an immunophilin-binding immunosuppressive agent.

12. A product as described in paragraph 11 wherein the immunosuppressive agent is slected from cyclosporin or a cyclosporin derivative, rapamycin or a rapamycin derivative, or FK506 or an FK506 derivative.

13. A product as described in paragraph 10 wherein the steroid or steroid derivative is selected from aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol or triamcinolone, and their respective pharmaceutically acceptable salts or derivatives.

14. A product as described in any one of the preceding paragraphs for the treatment of allergy, graft rejection, graft-versus host disease or autoimmune disease.

15. A method for preparing a product as described in any one of paragraphs 1 to 14 by combining:
i) a modulator of the Notch signalling pathway; and
ii) an immunosuppressive agent.

16. A method for modulating the immune system in a mammal comprising simultaneously, contemporaneously, separately or sequentially administering:
i) an effective amount of a modulator of the Notch signalling pathway; and
ii) an effective amount of an immunosuppressive agent.

17. A method for modulation of the immune system comprising contacting a T cell with a stimulatory signal and an appropriate amount of a combination of a modulator of Notch signalling and an immunosuppressive agent, to promote differentiation to a regulatory T cell.

18. A combination of:
i) a modulator of the Notch signalling pathway; and
ii) an immunosuppressive agent;
for simultaneous, contemporaneous, separate or sequential use in modulating the immune system.

19. A modulator of the Notch signalling pathway for use in modulating the immune system in simultaneous, contemporaneous, separate or sequential combination with an immunosuppressive agent.

20. The use of a combination of:
i) a modulator of the Notch signalling pathway; and
ii) an immunosuppressive agent;
in the manufacture of a medicament for modulation of the immune system.

21. The use of a modulator of the Notch signalling pathway in the manufacture of a medicament for modulation of the immune system in simultaneous, contemporaneous, separate or sequential combination with an immunosuppressive agent.

22. A pharmaceutical kit comprising a modulator of the Notch signalling pathway and an immunosuppressive agent.

23. A pharmaceutical composition comprising a modulator of the Notch signalling pathway and an immunosuppressive agent and optionally a pharmaceutically acceptable carrier.

24. A method for modulating the immune system, comprising the steps of:
administering an effective amount of a modulator of Notch signalling in a first treatment procedure; and administering an effective amount of an immunosuppressive agent in a second treatment procedure.

25. A method for modulating the immune system, comprising the steps of:
administering a synergistically effective amount of a modulator of Notch signalling in a first treatment procedure; and administering a synergistically effective amount of an immunosuppressive agent in a second treatment procedure.

26. A product comprising:
i) a modulator of the Notch signalling pathway; and
ii) an immunophilin-binding immunosuppressive agent;
as a combined preparation for simultaneous, contemporaneous, separate or sequential use for modulation of the immune system.

27. A product as described in paragraph 26 for modulation of T cell activity.

28. A product as described in paragraph 26 or paragraph 27 for the treatment of inflammation, asthma, allergy, graft rejection, graft-versus-host disease or autoimmune disease.

29. A product as described in any one of paragraphs 26 to 28 wherein the modulator of the Notch signalling pathway comprises Delta or Jagged or a fragment, derivative, homologue, analogue or allelic variant thereof or a polynucleotide coding for Delta, Jagged or a fragment, derivative, homologue, analogue or allelic variant thereof.

30. A product as described in any one of paragraphs 26 to 29 wherein the modulator of the Notch signalling pathway comprises a fusion protein comprising a segment of a Notch ligand extracellular domain and an immunoglobulin F_{c} segment or a polynucleotide coding for such a fusion protein.

31. A product as described in any one of paragraphs 26 to 30 wherein the modulator of the Notch signalling pathway comprises a protein or polypeptide comprising a DSL or EGF-like domain or a polynucleotide sequence coding for such a protein.

32. A product as described in paragraph 31 wherein the modulator of the Notch signalling pathway comprises a protein or polypeptide comprising a Notch ligand DSL domain at least one EGF-like domain or a polynucleotide sequence coding for such a protein or polypeptide.

33. A product as described in any one of paragraphs 26 to 28 wherein modulator of the Notch signalling pathway comprises Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

34. A product as described in any one of paragraphs 26 to 28 wherein the modulator of the Notch signalling pathway comprises a dominant negative version of a Notch signalling repressor, or a polynucleotide which codes for a dominant negative version of a Notch signalling repressor.

35. A product as described in any one of paragraphs 26 to 34 wherein the immunophilin-binding immunosuppressive agent is selected from cyclosporin or a cyclosporin derivative, rapamycin or a rapamycin derivative, or FK506 or an FK506 derivative.

36. A product as described in any one of paragraphs 26 to 35 for the treatment of allergy, graft rejection, graft-versus host disease or autoimmune disease.

37. A method for preparing a product as described in any one of paragraphs 26 to 36 by combining:
i) a modulator of the Notch signalling pathway; and
ii) an immunophilin-binding immunosuppressive agent.

38. A method for modulating the immune system in a mammal comprising simultaneously, contemporaneously, separately or sequentially administering:
i) an effective amount of a modulator of the Notch signalling pathway; and
ii) an effective amount of an immunophilin-binding immunosuppressive agent.

39. A method for modulation of the immune system comprising contacting a T cell with a stimulatory signal and an appropriate amount of a combination of a modulator of Notch signalling and an immunophilin-binding immunosuppressive agent, to promote differentiation to a regulatory T cell.

40. A combination of:
i) a modulator of the Notch signalling pathway; and
ii) an immunophilin-binding immunosuppressive agent;
for simultaneous, contemporaneous, separate or sequential use in modulating the immune system.

41. A modulator of the Notch signalling pathway for use in modulating the immune system in simultaneous, contemporaneous, separate or sequential combination with an immunophilin-binding immunosuppressive agent.

42. The use of a combination of:
i) a modulator of the Notch signalling pathway; and
ii) an immunophilin-binding immunosuppressive agent;
in the manufacture of a medicament for modulation of the immune system.

43. The use of a modulator of the Notch signalling pathway in the manufacture of a medicament for modulation of the immune system in simultaneous, contemporaneous, separate or sequential combination with an immunophilin-binding immunosuppressive agent.

44. A pharmaceutical kit comprising a modulator of the Notch signalling pathway and an immunophilin-binding immunosuppressive agent.

45. A pharmaceutical composition comprising a modulator of the Notch signalling pathway and an immunophilin-binding immunosuppressive agent and optionally a pharmaceutically acceptable carrier.

46. A method for modulating the immune system, comprising the steps of:
administering an effective amount of a modulator of Notch signalling in a first treatment procedure; and administering an effective amount of an immunophilin-binding immunosuppressive agent in a second treatment procedure.

47. A method for modulating the immune system, comprising the steps of:
administering a synergistically effective amount of a modulator of Notch signalling in a first treatment procedure; and administering a synergistically effective amount of an immunophilin-binding immunosuppressive agent in a second treatment procedure.

48. A product comprising:
i) a modulator of the Notch signalling pathway; and
ii) an immunologically active steroid;
as a combined preparation for simultaneous, contemporaneous, separate or sequential use for modulation of the immune system.

49. A product as described in paragraph 48 for modulation of T cell activity.

50. A product as described in paragraph 48 or paragraph 49 for the treatment of inflammation, asthma, allergy, graft rejection, graft-versus-host disease or autoimmune disease.

51. A product as described in any one of paragraphs 48 to 50 wherein the modulator of the Notch signalling pathway comprises Delta or Jagged or a fragment, derivative, homologue, analogue or allelic variant thereof or a polynucleotide coding for Delta, Jagged or a fragment, derivative, homologue, analogue or allelic variant thereof.

52. A product as described in any one of paragraphs 48 to 51 wherein the modulator of the Notch signalling pathway comprises a fusion protein comprising a segment of a Notch ligand extracellular domain and an immunoglobulin F_{c} segment or a polynucleotide coding for such a fusion protein.

53. A product as described in any one of paragraphs 48 to 52 wherein the modulator of the Notch signalling pathway comprises a protein or polypeptide comprising a DSL or EGF-like domain or a polynucleotide sequence coding for such a protein.

54. A product as described in paragraph 53 wherein the modulator of the Notch signalling pathway comprises a protein or polypeptide comprising a Notch ligand DSL domain at least one EGF-like domain or a polynucleotide sequence coding for such a protein or polypeptide.

55. A product as described in any one of paragraphs 48 to 50 wherein modulator of the Notch signalling pathway comprises Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

56. A product as described in any one of paragraphs 48 to 50 wherein the modulator of the Notch signalling pathway comprises a dominant negative version of a Notch signalling repressor, or a polynucleotide which codes for a dominant negative version of a Notch signalling repressor.

57. A product as described in any of paragraphs 48 to 56 wherein the immunologically active steroid or steroid derivative is selected from aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol or triamcinolone, and their respective pharmaceutically acceptable salts or derivatives.

58. A product as described in any one of paragraphs 48 to 57 for the treatment of allergy, graft rejection, graft-versus host disease or autoimmune disease.

59. A method for preparing a product as described in any one of paragraphs 48 to 58 by combining:
i) a modulator of the Notch signalling pathway; and
ii) an immunosuppressive agent.

60. A method for modulating the immune system in a mammal comprising simultaneously, contemporaneously, separately or sequentially administering:
i) an effective amount of a modulator of the Notch signalling pathway; and
ii) an effective amount of an immunologically active steroid or steroid derivative.

61. A method for modulation of the immune system comprising contacting a T cell with a stimulatory signal and an appropriate amount of a combination of a modulator of Notch signalling and an immunologically active steroid or steroid derivative, to promote differentiation to a regulatory T cell.

62. A combination of:
i) a modulator of the Notch signalling pathway; and
ii) an immunologically active steroid or steroid derivative;
for simultaneous, contemporaneous, separate or sequential use in modulating the immune system.

63. A modulator of the Notch signalling pathway for use in modulating the immune system in simultaneous, contemporaneous, separate or sequential combination with an immunologically active steroid or steroid derivative.

64. The use of a combination of:
i) a modulator of the Notch signalling pathway; and
ii) an immunologically active steroid or steroid derivative;
in the manufacture of a medicament for modulation of the immune system.

65. The use of a modulator of the Notch signalling pathway in the manufacture of a medicament for modulation of the immune system in simultaneous, contemporaneous, separate or sequential combination with an immunologically active steroid or steroid derivative.

66. A pharmaceutical kit comprising a modulator of the Notch signalling pathway and an immunologically active steroid or steroid derivative.

67. A pharmaceutical composition comprising a modulator of the Notch signalling pathway and an immunologically active steroid or steroid derivative and optionally a pharmaceutically acceptable carrier.

68. A method for modulating the immune system, comprising the steps of: administering an effective amount of a modulator of Notch signalling in a first treatment procedure; and administering an effective amount of an immunologically active steroid or steroid derivative in a second treatment procedure.

69. A method for modulating the immune system, comprising the steps of: administering a synergistically effective amount of a modulator of Notch signalling in a first treatment procedure; and administering a synergistically effective amount of an immunologically active steroid or steroid derivative in a second treatment procedure.

### REFERENCES

Artavanis-Tsakonas S, et al. (1995) Science 268:225-232.
Artavanis-Tsakonas S, et al. (1999) Science 284:770-776.
Brucker K, et al. (2000) Nature 406:411-415.
Camilli et al. (1994) Proc Natl Acad Sci USA 91:2634-2638.
Chee M. et al. (1996) Science 274:601-614.
Dkuz (1997) Cell 90: 271-280
Hicks C, et al. (2000) Nat. Cell. Biol. 2:515-520.
Hoyne G. F. et al (1999) Int Arch Allergy Immunol 118:122-124;
Hoyne et al. (2000) Immunology 100:281-288;
Hoyne G. F. et al (2000) Intl Immunol 12:177-185;
Hoyne, G. et al. (2001) Immunological Reviews 182:215-227
Irvine K D (1999) Curr. Opin. Genet. Devel. 9:434-441.
Ju B J, et al. (2000) Nature 405:191-195.
Leimeister C. et al. (1999) Mech Dev 85 (1-2):173-7.
Li et al. (1998) Immunity 8 (1):43-55.
Lieber, T. et al. (1993) Genes Dev 7 (10):1949-65.
Lu, F. M. et al. (1996) Proc Natl Acad Sci 93 (11):5663-7.
Matsuno K, et al. (1998) Nat. Genet. 19:74-78.
Matsuno, K. et al. (1995) Development 121 (8):2633-44.
Medhzhitov et al. (1997) Nature 388:394-397.
McGuiness (1996) Genomics 35 (3): 473-485
Meuer S. et al (2000) Rapid Cycle Real-time PCR, Springer-Verlag Berlin and Heidelberg GmbH & Co.
Moloney D J, et al. (2000) Nature 406:369-375.
Munro S, Freeman M. (2000) Curr. Biol. 10:813-820.
Ordentlich et al. (1998) Mol. Cell. Biol. 18:2230-2239.
Osborne B, Miele L. (1999) Immunity 11:653-663.
Panin V M, et al. (1997) Nature 387:908-912.
Schroeter E H, et al. (1998) Nature 393:382-386.
Struhl G, Adachi A. (1998) Cell 93:649-660.
Struhl, G. et al. (1998) Cell 93 (4):649-60.
Takebayashi K. et al. (1994) J Biol Chem 269 (7):150-6.
Tamura K, et al. (1995) Curr. Biol. 5:1416-1423.
Valenzuela et al. (1995) J. Neurosci. 15:6077-6084.
Weinmaster G. (2000) Curr. Opin. Genet. Dev. 10:363-369.
Wilson and Hemmati-Brivanlou (1997) Neuron 18:699-710.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in

We claim:

1. A composition for reducing an immune response comprising:
i) an agonist of Notch signalling; and
ii) an immunosuppressive agent.

2. The composition as claimed in claim 1, wherein the agonist of Notch signalling comprises a molecule selected from the group consisting of (i) Delta; (ii) Jagged; (iii) a fragment, derivative, homologue, analogue or allelic variant of Delta or Jagged; (iv) a fusion protein comprising a segment of a Notch ligand extracellular domain and an immunoglobulin Fc segment; (v) a protein or polypeptide comprising a DSL or EGF-like domain; (vi) Notch intracellular domain (Notch IC) or a fragment, derivative, homologue, analogue or allelic variant thereof; (vii) a dominant negative version of a Notch signalling repressor; and (viii) a polynucleotide sequence encoding any one of (i) to (vii).

3. The composition as claimed in claim 1, wherein the agonish of Notch signalling comprises a protein or polypeptide comprising a Notch ligand DSL domain at least one EGF-like domain, or a polynucleotide sequence coding for the protein or polypeptide.

4. The composition as claimed in claim 1, wherein the immunosuppressive agent is selected from the group consisting of an immunologically active steroid or steroid derivative; an immunophilin-binding immunosuppressive agent; cyclosporin or a cyclosporin derivative; rapamycin or a rapamycin derivative; and FK506 or an FK506 derivative.

5. The composition as claimed in claim 1, wherein the immunosuppressive agent is an immunophilin-binding immunosuppressive agent.

6. The composition as claimed in claim 1, wherein the immunosuppressive agent is an immunologically active steroid or steroid derivative.

7. The composition as claimed in claim 6, wherein the steroid or steroid derivative is selected from the group consisting of aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol or triamcinolone, and their respective pharmaceutically acceptable salts or derivatives.

8. A method for reducing an immune response in a mammal comprising simultaneously, contemporaneously, separately or sequentially administering to the mammal:
i) an effective amount of an agonist of Notch signalling; and
ii) an effective amount of an immunosuppressive agent.

9. The method as claimed in claim 8, wherein reducing an immune response treats inflammation, asthma, allergy, graft rejection, graft-versus-host disease or autoimmune disease.

10. The method as claimed in claim 8, wherein the agonist of Notch signalling is administered to the mammal in a first treatment procedure; and the immunosuppressive agent is administered to the mammal in a second treatment procedure.

11. The method as claimed in claim 8, wherein a synergistic amount of the agonist of Notch signalling is administered to the mammal in a first treatment procedure; and a synergistic amount of the immunosuppressive agent is administered to the mammal in a second treatment procedure.

12. The method as claimed in claim 8, wherein the agonist of Notch signalling comprises a molecule selected from the group consisting of (i) Delta; (ii) Jagged; (iii) a fragment, derivative, homologue, analogue or allelic variant of Delta or Jagged; (iv) a fusion protein comprising a segment of a Notch ligand extracellular domain and an immunoglobulin F_{c} segment; (v) a protein or polypeptide comprising a DSL or EGF-like domain; (vi) Notch intracellular domain (Notch IC) or a fragment, derivative, homologue, analogue or allelic variant thereof; (vii) a dominant negative version of a Notch signalling repressor; and (viii) a polynucleotide sequence encoding any one of (i) to (vii).

13. The method as claimed in claim 8, wherein the agonist of Notch signalling comprises a protein or polypeptide comprising a Notch ligand DSL domain at least one EGF-like domain, or a polynucleotide sequence coding for the protein or polypeptide.

14. The method as claimed in claim 8, wherein the immunosuppressive agent is selected from the group consisting of an immunologically active steroid or steroid derivative; an immunophilin-binding immunosuppressive agent; cyclosporin or a cyclosporin derivative; rapamycin or a rapamycin derivative; and FK506 or an FK506 derivative.

15. The method as claimed in claim 8, wherein the immunosuppressive agent is an immunophilin-binding immunosuppressive agent.

16. The method as claimed in claim 8, wherein the immunosuppressive agent is an immunologically active steroid or steroid derivative.

17. The method as claimed in claim 16, wherein the steroid or steroid derivative is selected from the group consisting of aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol or triamcinolone, and their respective pharmaceutically acceptable salts or derivatives.

18. A method for preparing the composition as claimed in claim 1 comprising combining:
i) an agonist of Notch signalling; and
ii) an immunosuppressive agent.

19. A method for promoting differentiation of a T cell to a regulatory T cell comprising contacting the T cell with a stimulatory signal and an effective amount of the composition as claimed in claim 1, whereby differentiation is promoted.

20. A kit comprising an agonist of Notch signalling and an immunosuppressive agent wherein the agonist and the agent are in the same or separate containers.


SEQUENCE LISTING (29 sequences)

SEQ ID NO: 1 - protein, 63 aa, Drosophila melanogaster
WKTNKSESQYTSLEYDFRVTCDLNYYGSGCAKFCRPRDDSFGHSTCSETGEIICLTGWQGDYC

SEQ ID NO: 2 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 3 - protein, 63 aa, Mus musculus
WSQDLHSSGRTDLRYSYRFVCDEHYYGEGCSVFCRPRDDAFGHFTCGDRGEKMCDPGWKGQYC

SEQ ID NO: 4 - protein, 63 aa, Rattus norvegicus
WSQDLHSSGRTDLRYSYRFVCDEHYYGEGCSVFCRPRDDAFGHFTCGERGEKMCDPGWKGQYC

SEQ ID NO: 5 - protein, 63 aa, Mus musculus
WRTDEQNDTLTRLSYSYRVICSDNYYGESCSRLCKKRDDHFGHYECQPDGSLSCLPGWTGKYC

SEQ ID NO: 6 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 7 - protein, 63 aa, Rattus norvegicus
WQTLKQNTGIAHFEYQIRVTCDDHYYGFGCNKFCRPRDDFFGHYACDQNGNKTCMEGWMGPEC

SEQ ID NO: 8 - protein, 63 aa, Mus musculus
WQTLKQNTGIAHFEYQIRVTCDDHYYGFGCNKFCRPRDDFFGHYACDQNGNKTCMEGWMGPDC

SEQ ID NO: 9 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 10 - protein, 63 aa, Gallus gallus
WQTLKHNTGAAHFEYQIRVTCAEHYYGFGCNKFCRPRDDFFTHHTCDQNGNKTCLEGWTGPEC

SEQ ID NO: 11 - protein, 63 aa, Gallus gallus
WKTLQFNGPVANFEVQIRVKCDENYYSALCNKFCGPRDDFVGHYTCDQNGNKACMEGWMGEEC

SEQ ID NO: 12 - protein, 63 aa, Mus musculus
WKSLHFSGHVAHLELQIRVRCDENYYSATCNKFCRPRNDFFGHYTCDQYGNKACMDGWMGKEC

SEQ ID NO: 13 - protein, 63 aa, Homo sapiens; identical to SEQ ID NO: 12

SEQ ID NO: 14 - protein, 63 aa, Rattus norvegicus; identical to SEQ ID NO: 12

SEQ ID NO: 15 - protein, 63 aa, Homo sapiens; identical to SEQ ID NO: 12

SEQ ID NO: 16 - protein, 63 aa, Drosophila melanogaster
WKTLDHIGRNARITYRVRVQCAVTYYNTTCTTFCRPRDDQFGHYACGSEGQKLCLNGWQGVNC

SEQ ID NO: 17 - protein, 723 aa, Homo sapiens [sequence omitted]

SEQ ID NO: 18 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 19 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 20 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 21 - protein, 1238 aa, Homo sapiens [sequence omitted]

SEQ ID NO: 22 - DNA, 3162 bp, Homo sapiens
gaattccattttaagttatacaaaactgattaccataagtgcggtcgactgcttttatttttacgttgtgtgtgttggaaaaatgctaaaacatcagtctacaattctatatattgttattaaagattaatccaaccagcaacccaaggacatataagcgatttccactattgcatcagagcactcggcaggaaaggcctagccacggggaacattagaagctacagaagcattgcagagaagagaagatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccacggctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaggaattc

SEQ ID NO: 23 - DNA, 5590 bp, Homo sapiens
ctgcggccggcccgcgagctaggctgggtttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtatggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattacgagaacaagaactccaaaatgtctaaaataaggacacacaattctgaagtagaagaggacgacatggacaaacaccagcagaaagcccggtttgccaagcagccggcgtatacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttatttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaattttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatctgactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaac

SEQ ID NO: 24 - protein, 332 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDEC

SEQ ID NO: 25 - protein, 43 aa, Artificial Sequence
  Description of Artificial Sequence Synthetic consensus sequence
CXXXXXXXXCXXXCXXXXXXXXXXXCXXXXXXXCXXXXXXXXC

SEQ ID NO: 26 - protein, 43 aa, Artificial Sequence; identical to SEQ ID NO: 25
  Description of Artificial Sequence Synthetic consensus sequence

SEQ ID NO: 27 - protein, 43 aa, Artificial Sequence
  Description of Artificial Sequence Synthetic consensus sequence
CXXXYYXXXCXXXCRPRXDXFGHXXCXXXGXXXCXXGWXGXXC

SEQ ID NO: 28 - protein, 175 aa, Artificial Sequence
  Description of Artificial Sequence Synthetic EGF-like domain
XXXXCXXXXXXXXXXXXXXXXXXXXXXXXXXXXXXXXXXXXXXXXXXXXXXXXCXXXXXXXXXXXXCXXXXXXXXXXXXXXXXXXXXXXXXXXXXXXXXXXXXXXXXXXXXXXXXXXXXXXXXXXXXXXXXXXXXXXCXXXXXXCXXGXXXXXXXXXXXXXXXXXXXXXXGXXCX

SEQ ID NO: 29 - protein, 864 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK